(19) United States
(12) Patent Application Publication
(10) Pub. No.: US 2022/0000888 A1
(43) Pub. Date: Jan. 6, 2022

(54) OPHTHALMIC COMPOSITIONS

(71) Applicant: Aciont Inc., Salt Lake City, UT (US)

(72) Inventors: John W. Higuchi, Salt Lake City, UT (US); Kongnara Papangkorn, Sandy, UT (US)

(73) Assignee: Aciont Inc., Salt Lake City, UT (US)

(21) Appl. No.: 17/359,120

(22) Filed: Jun. 25, 2021

Related U.S. Application Data
(63) Continuation of application No. 16/155,641, filed on Oct. 9, 2018, now abandoned.
(60) Provisional application No. 62/569,430, filed on Oct. 6, 2017.

(51) Int. Cl. A61K 31/661; A61K 9/00; A61F 9/00; A61P 37/06; A61P 27/02

(52) U.S. Cl. CPC A61K 31/661; A61K 9/0048; A61P 27/02; A61P 37/06; A61F 9/0017

(57) ABSTRACT

An ophthalmic composition can include dexamethasone phosphate, or a salt thereof, dexamethasone, but in an amount not greater than 1.0 wt % relative to the amount of dexamethasone phosphate, or a salt thereof, and water. The ophthalmic composition can have a pH of about 5 to about 8 and a tonicity of from about 100 mOsm/kg to about 760 mOsm/kg.

### RELATED APPLICATION

This application is a continuation of U.S. patent application Ser. No. 16/155,641, filed Oct. 9, 2018, which claims the benefit of U.S. Provisional Patent Application Ser. No. 62/569,430, filed Oct. 6, 2017, each of which is incorporated herein by reference.

### BACKGROUND

Ocular administration of steroids can be very effective at treating a number of ocular conditions, but the ophthalmic side effects of steroids can be substantial. For example, ocular administration of steroids can lead to cataracts, glaucoma, secondary infection, and/or delayed healing of the ocular condition. However, these adverse effects are typically manageable. Thus, steroids are generally warranted when a patient has a vision-threatening condition.

### BRIEF DESCRIPTION OF THE DRAWINGS

For a fuller understanding of the nature and advantage of the present invention, reference is being made to the following detailed description of preferred embodiments and in connection with the accompanying drawings, in which:

FIG. 1a illustrates a front cross-sectional view of a non-invasive ocular drug delivery device, in accordance with some examples of the present disclosure.

FIG. 1b illustrates a bottom view of the non-invasive ocular drug delivery device of FIG. 1a.

FIG. 2a illustrates a front cross-sectional view of a non-invasive ocular drug delivery device, in accordance with other examples of the present disclosure.

FIG. 2b illustrates a bottom perspective view of the non-invasive ocular drug delivery device of FIG. 2a.

FIG. 2c illustrates a bottom view of the non-invasive ocular drug delivery device of FIG. 2c.

FIG. 3a illustrates a perspective view of a non-invasive ocular drug delivery device, in accordance with yet other examples of the present disclosure.

FIG. 3b illustrates a side cross-sectional view of the non-invasive ocular drug delivery device of FIG. 3a.

FIG. 3c illustrates a top view of the non-invasive ocular drug delivery device of FIG. 3a.

FIG. 3d illustrates a bottom view of the non-invasive ocular drug delivery device of FIG. 3a.

FIG. 4 illustrates a side cross-sectional view of the device of FIG. 3a attached to an eye, in accordance with some examples of the present disclosure.

FIG. 5 is a graph of vitreous scores of various treatment groups tested in an experimental uveitis rabbit model.

FIG. 6a is a magnified image of a posterior section of an untreated eye depicting severe inflammation and damaged photoreceptor layer (arrow).

FIG. 6b is a magnified image of a posterior section of an eye treated with 15% DSP (15 minutes, 4 doses) depicting minimal inflammation and well-preserved tissue structure.

FIG. 7 is a graph (mean±SD, n=6 eyes) of the amount of drug in the eye, application time, and DSP concentration after single administration of drug via a non-invasive ocular drug delivery device.

FIG. 8a is a graph of mean plasma concentration of DSP (solid line) and DEX (dotted line) following single administration of DSP via a non-invasive ocular drug delivery device.

FIG. 8b is a graph of mean plasma concentration of DSP equivalent following single administration of DSP via a non-invasive ocular drug delivery device. The data were calculated from FIG. 10a based on the sum of DSP and DEX in gram equivalent. No standard deviation is given. To reveal all pharmacokinetic data, graph was not plotted in a linear time sale on the x-axis.

### DESCRIPTION OF EMBODIMENTS

Although the following detailed description contains many specifics for the purpose of illustration, a person of ordinary skill in the art will appreciate that many variations and alterations to the following details can be made and are considered to be included herein. Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this written description, the singular forms “a,” “an” and “the” include express support for plural referents unless the context clearly dictates otherwise. Thus, for example, reference to “a polymer” can include a plurality of such polymers.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” in this written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, the term “threshold dose” refers to an amount of a therapeutic agent which, when administered to a subject, is sufficient to achieve an intended therapeutic or physiological effect. Thus, a “threshold dose” refers to a non-toxic, but sufficient dose of a therapeutic agent, to achieve therapeutic results in treating a condition for which the therapeutic agent is known to be effective. It is understood that various biological factors may affect the ability of a therapeutic agent to perform its intended task. Therefore, a “threshold dose” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of a threshold dose is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this written description support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Reference in this application may be made to compositions, systems, or methods that provide “improved” or “enhanced” performance. It is to be understood that unless otherwise stated, such “improvement” or “enhancement” is a measure of a benefit obtained based on a comparison to compositions, systems or methods in the prior art. Furthermore, it is to be understood that the degree of improved or enhanced performance may vary between disclosed embodiments and that no equality or consistency in the amount, degree, or realization of improvement or enhancement is to be assumed as universally applicable.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### Example Embodiments

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

The present disclosure is directed to ophthalmic compositions suitable for treating a number of ophthalmic conditions in a subject. The ophthalmic composition can include dexamethasone phosphate, or a salt thereof (e.g. sodium salt, for example). Additionally, the ophthalmic composition can include dexamethasone. However, dexamethasone is typically not present in an amount greater than 0.5 wt % relative to dexamethasone phosphate, or a salt thereof. The ophthalmic composition can also include water. The pH of the composition can typically be from about 5 to about 8 and the tonicity can typically be from about 300 mOsm/kg to about 760 mOsm/kg.

In further detail, the dexamethasone phosphate, or salt thereof, can typically be present in the composition in a variety of amounts. For example, in some cases, the dexamethasone phosphate, or salt thereof, can be present in the composition in an amount from about 1 wt % to about 25 wt %. In some examples, the dexamethasone phosphate, or salt thereof, can be present in the composition in an amount greater than 4 wt %. In some examples, the dexamethasone phosphate, or salt thereof, can be present in the composition in an amount greater than 10 wt %. In some examples, the dexamethasone phosphate, or salt thereof, can be present in the composition in an amount from about 1 wt % to about 3 wt %. In other examples, the dexamethasone phosphate, or salt thereof, can be present in the composition in an amount from about 3 wt % to about 6 wt %. In some other examples, the dexamethasone phosphate, or salt thereof, can be present in the composition in an amount from about 6 wt % to about 10 wt %. In still other examples, the dexamethasone phosphate, or salt thereof, can be present in the composition in an amount from about 10 wt % to about 15 wt %. In yet other examples, the dexamethasone phosphate, or salt thereof, can be present in the composition in an amount from about 12 wt % to about 18 wt %.

It is also noted that the carrier of the ophthalmic composition can provide good stability for the dexamethasone phosphate, or salt thereof. For example, in some cases, the amount of dexamethasone present in the ophthalmic composition does not exceed or is not more than 0.5 wt % relative to dexamethasone phosphate, or salt thereof, after storage at ambient temperature for a period of 3 months or less. In some examples, the amount of dexamethasone present in the ophthalmic composition does not exceed or is not more than 1.0 wt % relative to dexamethasone phosphate, or salt thereof, after storage at ambient temperature for a period of 3 months or less. In other examples, the amount of dexamethasone present in the ophthalmic composition does not exceed or is not more than 0.5 wt % relative to dexamethasone phosphate, or salt thereof, after storage at ambient temperature for a period of 6 months or less. In some examples, the amount of dexamethasone present in the ophthalmic composition does not exceed or is not more than 1.0 wt % relative to dexamethasone phosphate, or salt thereof, after storage at ambient temperature for a period of 6 months or less. In other examples, the amount of dexamethasone present in the ophthalmic composition does not exceed or is not more than 1.0 wt % relative to dexamethasone phosphate, or salt thereof, after storage at ambient temperature for a period of 9 months or less.

In additional examples, the amount of dexamethasone present in the ophthalmic composition does not exceed or is not more than 0.5 wt % relative to dexamethasone phosphate, or salt thereof, after storage a temperature of from about 2° C. to about 8° C. for a period of 6 months or less. In some examples, the amount of dexamethasone present in the ophthalmic composition does not exceed or is not more than 1.0 wt % relative to dexamethasone phosphate, or salt thereof, after storage a temperature of from about 2° C. to about 8° C. for a period of 6 months or less. In other examples, the amount of dexamethasone present in the ophthalmic composition does not exceed or is not more than 0.5 wt % relative to dexamethasone phosphate, or salt thereof, after storage a temperature of from about 2° C. to about 8° C. for a period of 12 months or less. In some examples, the amount of dexamethasone present in the ophthalmic composition does not exceed or is not more than 1.0 wt % relative to dexamethasone phosphate, or salt thereof, after storage a temperature of from about 2° C. to about 8° C. for a period of 12 months or less. In some other examples, the amount of dexamethasone present in the ophthalmic composition does not exceed or is not more than 0.5 wt % relative to dexamethasone phosphate, or salt thereof, after storage a temperature of from about 2° C. to about 8° C. for a period of 18 months or less. In some examples, the amount of dexamethasone present in the ophthalmic composition does not exceed or is not more than 1.0 wt % relative to dexamethasone phosphate, or salt thereof, after storage a temperature of from about 2° C. to about 8° C. for a period of 18 months or less. In additional examples, the amount of dexamethasone present in the ophthalmic composition does not exceed or is not more than 0.5 wt % relative to dexamethasone phosphate, or salt thereof, after storage a temperature of from about 2° C. to about 8° C. for a period of 24 months or less. In some examples, the amount of dexamethasone present in the ophthalmic composition does not exceed or is not more than 1.0 wt % relative to dexamethasone phosphate, or salt thereof, after storage a temperature of from about 2° C. to about 8° C. for a period of 24 months or less. In yet other examples, the amount of dexamethasone present in the ophthalmic composition does not exceed or is not more than 0.5 wt % relative to dexamethasone phosphate, or salt thereof, after storage a temperature of from about 2° C. to about 8° C. for a period of 36 months or less. In some examples, the amount of dexamethasone present in the ophthalmic composition does not exceed or is not more than 1.0 wt % relative to dexamethasone phosphate, or salt thereof, after storage a temperature of from about 2° C. to about 8° C. for a period of 36 months or less. In still further examples, the amount of dexamethasone present in the ophthalmic composition does not exceed or is not more than 0.5 wt % relative to dexamethasone phosphate, or salt thereof, after storage a temperature of from about 2° C. to about 8° C. for a period of 49 months or less. In some examples, the amount of dexamethasone present in the ophthalmic composition does not exceed or is not more than 1.0 wt % relative to dexamethasone phosphate, or salt thereof, after storage a temperature of from about 2° C. to about 8° C. for a period of 49 months or less.

The ophthalmic composition can generally have a pH of from about 5 to about 8. However, in some examples, the ophthalmic composition can have a pH of from about 6 to about 9. In other examples, the pH can be from about 6.5 or 6.6 to about 7.4 or 7.5. In some examples, the ophthalmic composition can include a suitable pH adjuster, such as hydrochloric acid, phosphoric acid, sodium hydroxide, the like, or a combination thereof.

The tonicity of the ophthalmic composition can be influenced by a number of factors, such as the amount of dexamethasone phosphate, or salt thereof, present in the composition, the presence of a tonicity agent, etc. The tonicity of the ophthalmic composition can typically be from about 300 mOsm/kg to about 760 mOsm/kg. In some specific examples, the tonicity can be from about 300 mOsm/kg to about 350 mOsm/kg. In yet other examples, the tonicity of the composition can be from about 325 mOsm/kg to about 375 mOsm/kg or 400 mOsm/kg. In still other examples, the tonicity can be from about 400 mOsm/kg to about 500 mOsm/kg. In yet additional examples, the tonicity can be from about 500 mOsm/kg to about 600 mOsm/kg. In some examples, the composition can include a tonicity agent. Non-limiting examples can include sodium chloride, potassium chloride, calcium chloride, magnesium chloride, mannitol, sorbitol, dextrose, glycerin, propylene glycol, ethanol, trehalose, the like, or combinations thereof. In some examples, where the active agent is dexamethaxone sodium phosphate (DSP), the amount of DSP present in the composition can provide the desired tonicity without the need for an additional tonicity agent. As such, in some examples, the composition does not include a tonicity agent.

In some specific examples, the composition can include a chelating agent. Non-limiting examples can include edetate disodium dihydrate, edetic acid, ethylene diamine, porphine, the like, or combinations thereof. Where a chelating agent is included in the ophthalmic composition, it can typically be present in an amount of from about 0.001% w/v to about 0.1% w/v, or from about 0.005% w/v to about 0.05% w/v. However, in some examples, the composition does not include a chelating agent. For example, in some cases, dexamethasone, or salt thereof, can be sourced or prepared to have a very low concentration of metal ions. Alternatively, or additionally, in some examples, the container can impart a very low amount of metal ions to the composition. For example, in some cases, the container can be a plastic container, a glass container with an interior plastic coating, or the like, that does not impart significant amounts of metal ions. Thus, a chelating agent is not always desirable or necessary.

The ophthalmic composition can typically be particulate-matter-free or substantially particulate-matter-free. As used herein, the term “particulate-matter-free” or its grammatical equivalents such as “particle free” refer to the state in which the ophthalmic composition meets the USP requirements for particulate matter in ophthalmic compositions. See for example, USP, Chapter 789. One of skill in the art understands and knows how to assess whether a given composition meets USP particulate matter requirements. With this in mind, in some examples, the ophthalmic composition can include less than or equal to 50 particles per milliliter (mL) having a particle diameter greater than or equal to 10 μm. In still additional examples, the ophthalmic composition can include less than or equal to 5 particles per mL having a particle diameter greater than or equal to 25 μm. These values can be determined using the Light Obscuration Particle Count Test, the Microscopic Particle Count Test, or both, as described in USP, Chapter 789.

Thus, in some examples, the ophthalmic composition can be an ophthalmic solution. As such, in some examples, the ophthalmic composition is not a gel, an ointment, a suspension, an emulsion, or the like. Further, in some examples, the ophthalmic composition can include limited amounts of excipients. As used herein, an “excipient” refers to components in the ophthalmic composition other than dexamethasone phosphate, or salt thereof, and water. In some specific examples, the total amount of excipients in the ophthalmic composition can be less than 10% w/v. In some additional examples, the total amount of excipients in the ophthalmic composition can be less than 5% w/v, less than 3% w/v, or less than 1% w/v.

For example, in some cases, the ophthalmic composition can be free or substantially free of a preservative. Non-limiting examples of preservatives can include benzalkonium chloride, benzethonium chloride, parahydroxybenzoates, phenylmercuric acetate, cetrimonium, chlorobutanol, phenylethylalcohol, polyquaternium-1, polyhexamethylene biguanide, sodium perborate, stabilized oxychloro complex, and the like. In some examples, the ophthalmic composition is free or substantially free of an antioxidant. Non-limiting examples of antioxidants can include sodium metabisulphite, sodium formaldehyde sulphoxylate, sodium sulphite, N-acetylcarnosine, L-carnosine, L-glutathione, cysteine ascorbate, L-cysteine, and the like. In some additional examples, the ophthalmic composition can be free or substantially free of a buffering agent, such as phosphate-buffered saline, TRIS-buffered saline, and the like. In still additional examples, the composition can be free or substantially free of a polymer (e.g. a thickening agent, a gelling agent, or the like), such as cellulosic compounds (e.g. carboxymethyl cellulose, hydroxypropyl cellulose, hydroxyethyl cellulose, etc.), carbomers, polyvinyl alcohol, gelatin, polyvinyl pyrrolidone, polysaccharide thickeners (e.g. starches, vegetable gums, pectin, etc.), or the like, including combinations thereof. In some examples, the ophthalmic composition does not include a surface-active agent or surfactant. Non-limiting examples of surface-active agents can include non-ionic surfactants (e.g. sorbitan oleates, polysorbates, polyoxyethylene ethers, etc.) anionic surfactants, and cationic surfactants. In some examples, the ophthalmic composition does not include a cyclodextrin, or the like. In some examples, the ophthalmic composition does not include a hydrocarbon, a sterol (e.g. cholesterol), or both. In some examples, the ophthalmic composition does not include one or more of a preservative, an antioxidant, a buffering agent, a polymer, a surface-active agent, a cyclodextrin, a hydrocarbon, or a sterol. In some examples, the ophthalmic composition does not include two or more of a preservative, an antioxidant, a buffering agent, a polymer, a surface-active agent, a cyclodextrin, a hydrocarbon, or a sterol. In some examples, the ophthalmic composition does not include three or more of a preservative, an antioxidant, a buffering agent, a polymer, a surface-active agent, a cyclodextrin, a hydrocarbon, or a sterol. In some examples, the composition does not include one or more of a polymer, a dendrimer, a micelle, a liposome, a nanoparticle, a surface-active agent, or the like.

In some specific examples, the ophthalmic composition can consist of, or consist essentially of, dexamethasone phosphate, or a salt thereof, a chelating agent, water, optionally a tonicity agent, and optionally a pH adjuster. In some examples, the chelating agent can be disodium edetate. In some examples, the composition can include dexamethasone sodium phosphate (DSP).

In some examples, the ophthalmic composition can be sterile. A number of sterilization procedures can be used to sterilize the ophthalmic composition. Non-limiting examples of sterilization procedures can include EtO sterilization, gamma sterilization, E-beam sterilization, x-ray sterilization, vaporized hydrogen peroxide (VHP) sterilization, steam sterilization, dry-heat sterilization, filtration, the like, or combinations thereof.

In some examples, the ophthalmic composition can be supplied in an enclosed container, which can be a sterile container. Where this is the case, the container can include any suitable container. In some examples, the container can be made of a material such as glass, polyethylene, polypropylene, polyvinyl chloride, polycarbonate, the like, or a combination thereof. In some examples, the container can have a volume of from about 0.1 ml to about 10 ml, from about 0.5 ml to about 5 ml, or from about 0.75 ml to about 1.5 ml.

In some additional examples, the amount of active agent composition supplied in the container can include from about 1 mg to about 2500 mg active agent (e.g. dexamethasone phosphate, for example). In other examples, the amount of active agent composition supplied in the container can include from about 10 mg to about 50 mg, from about 50 mg to about 500 mg, from about 500 mg to about 1000 mg, or from about 1000 mg to about 2000 mg active agent.

In some embodiments, the concentration of active agent in the composition or solution can cause the composition or solution to turn from substantially colorless to colored. In one embodiment, the color of the solution with an active agent (e.g. dexamethasone phosphate) can be visibly yellow or yellowish. In one embodiment the composition or solution can have a color with a wavelength ranging from about 560 nm to about 590 nm. In other embodiments, the composition or solution can have a color with a frequency interval ranging from about 510 THz to about 540 THz. In some embodiments, the composition or solution can have color properties falling within both of the above-recited wavelength and frequency ranges, including any point or specific number in either and any combination thereof.

In some further examples, the ophthalmic composition can be further supplied with or in an ocular drug delivery device that is adapted to couple to an eye of a subject. Thus, some or all of the composition can be included in the ocular drug delivery device, the container, or a combination thereof. For example, in some cases, a portion of the active agent composition can be pre-loaded into the ocular drug delivery device, and another portion can be loaded into the container for use in re-loading the ocular drug delivery device after administration of the pre-loaded portion. In other examples, the active agent composition can be initially contained within the container for subsequent transfer to load the ocular drug delivery device. In still other examples, the ocular drug delivery device can be pre-loaded with the active agent composition and supplied without a separate container including additional active agent composition. In some specific examples, the device can include at least 50 μL, at least 100 μL, or at least 150 μL of the ophthalmic composition pre-loaded therein. In some examples, the device can include from about 50 μL to about 5000 μL of the ophthalmic composition pre-loaded therein. In other examples, the device can include from about 100 μL to about 1000 μL of the ophthalmic composition pre-loaded therein. In yet other examples, the device can include from about 150 μL to about 500 μL of the ophthalmic composition pre-loaded therein. In some specific examples, the device can include from about 120 μL to about 300 μL of the ophthalmic composition pre-loaded therein.

A variety of suitable devices can be used. In some specific examples, the device can be a non-invasive ocular drug delivery device including a housing and an active agent matrix coupled to the housing.

In further detail, the housing of the non-invasive ocular drug delivery device is not particularly limited, other than it is adapted to couple to an eye of a subject. Thus, in some examples, the housing can couple directly to the eye, such as via negative pressure, surface tension, adhesives, the like, or combinations thereof. In yet other examples, the housing can be shaped to interface with the eye and can be held against the eye using positive pressure from eye lids, and/or straps, cords, scaffolding, adhesives, the like, or combinations thereof that are attached to a surface outside of the eye, but nonetheless hold the housing in place against the eye.

In some examples, the housing can be formed from a plurality of interconnecting pieces to prepare an integral housing. In yet other examples, the housing can be formed as a monolithic unit. Thus, in some cases, the housing can be formed from a mold or other suitable manufacturing process as a single monolithic unit without any need for further assembly or integration of additional components. In some specific examples, the monolithic unit can be formed of a molded elastomeric material, such as ethylene propylene diene monomer (EPDM), fluoroelastomers (e.g. FKMs, FFKMs, FEPMs, etc.), acrylonitrile-butadiene rubbers, silicones, the like, or combinations thereof. Whether the housing is formed of a molded material or not, the housing can include a variety of suitable materials, such as one or more of the elastomeric materials listed above, polyamides, polyesters, polyethylenes, polypropylenes, polycarbonates, polyurethanes, polytetrafluoroethylenes, metals, the like, or combinations thereof. In some specific examples, the housing can include or be formed of an EPDM material. In yet other examples, the housing can include or be formed of a fluoroelastomer material. In still other examples, the housing can include or be formed of an acrylonitrile-butadiene rubber. In yet additional examples, the housing can include or be formed of a silicone material.

In still additional examples, the housing can include or be formed of a translucent or transparent material. For example, many of the materials listed above can be prepared in a way so that they are translucent or transparent. Other translucent or transparent materials can also be used. In some examples, portions of the housing can be translucent or transparent while others are not. In yet other examples, portions of the housing can be translucent while other parts of the housing can be transparent. In some specific examples, at least a portion of the housing that covers the cornea can be translucent or transparent.

The geometry of the housing is not particularly limited, so long as the housing adequately interfaces with a surface of the eye to facilitate administration of an active agent. However, in some examples, the housing (or at least the portion of the housing that interfaces with the eye) can have an elliptical geometry. While the overall shape of the eye approaches a spherical geometry, the part of the eye that is visible generally has an elliptical shape. Thus, the housing (or at least the portion of the housing that interfaces with the eye) can be prepared so as to have an elliptical, or approximately elliptical, shape. In some examples, an elliptical shape can facilitate application of the device to the eye and maximize the comfort of the subject, while maintaining adequate surface coverage or interface area of the device with the eye to provide an adequate dose of an active agent in a timely manner. Where the device has an elliptical geometry, the device can typically have an aspect ratio (width to height) of from about 1.05:1 to about 1.4:1. In yet other examples, the device can have an aspect ratio of from about 1.10:1 to about 1.3:1. In still other examples, the device can have an aspect ratio of from about 1.15:1 to about 1.25:1.

In some specific examples, the housing can include a corneal dome shaped to cover the cornea of the eye. The corneal dome can generally be shaped to maintain a gap between a portion of the cornea and an inner surface of the corneal dome. This gap can also facilitate the comfort of the user while using the device. As is known to one skilled in the art, the cornea can be a very sensitive portion of the eye. As such, in some cases, it can facilitate user comfort by minimizing contact of the device with the cornea. In some examples, the gap between the portion of the cornea and the inner surface of the corneal dome can be at least 50 μm or at least 100 μm. In yet other examples, the gap between the portion of the cornea and the inner surface of the corneal dome can be at least 200 μm or at least 500 μm. In still other examples, the gap between the portion of the cornea and the inner surface of the corneal dome can be at least 1000 μm. The portion of the cornea where the gap is maintained can generally be at least 50% of the corneal surface area. Thus, for example, in some cases, a gap of at least 100 μm between an inner surface of the corneal dome and the cornea of the eye can be maintained over at least 50% of the corneal surface. In some examples, the portion of the cornea where the gap is maintained can be at least 60%, 70%, 80%, or 90% of the corneal surface area. In yet other examples, the gap can be maintained across the entire corneal surface area.

In some additional examples, the housing can include a corneal seal that is positioned to circumscribe the cornea and form a fluidic seal against the eye to minimize fluid transport across the corneal seal to the cornea when in use. It is noted that where the device does not include a corneal dome, the cornea can be exposed to ambient conditions. However, the corneal seal can still minimize fluid transport (e.g. from the active agent matrix, for example) across the surface of the eye to the cornea. Where the housing includes a corneal dome, the corneal seal can be disposed about a periphery of the corneal dome to minimize fluid transport to the cornea when in use. It is noted that when the diameter of the corneal seal becomes too large, it can be challenging to comfortably maintain the housing within the framework of the eyelids. Thus, the corneal seal can be shaped to maintain a seal about the cornea without excessively increasing the overall size of the housing. In some examples, the corneal seal can be shaped to maintain a distance from a perimeter of the cornea (i.e. the corneal seal is positioned exterior to the cornea so as to not contact the cornea) of from about 50 μm to about 5000 μm. In yet other examples, the corneal seal can be shaped to maintain a distance from a perimeter of the cornea of from about 500 μm to about 3000 μm. In still other examples, the corneal seal can be shaped to maintain a distance from a perimeter of the cornea of from about 1000 μm to about 2000 μm. In some specific examples, the corneal seal can be shaped to maintain a distance from a perimeter of the cornea of from about 50 μm to about 1000 μm, about 100 μm to about 1500 μm, or about 300 μm to about 1200 μm.

In some further examples, the housing can include a scleral flange extending radially outward from the corneal seal. In some examples, where the housing (or at least the portion of the housing that interfaces with the eye) has an elliptical geometry, the scleral flange can have a shape that provides the elliptical geometry. In some examples, the scleral flange can be the portion of the housing to which the active agent matrix is attached. Where this is the case, the scleral flange can be shaped to maintain contact between the active agent matrix and the sclera of the eye when in use. The scleral flange can generally be shaped and positioned on the housing so as to cover a portion of the sclera of the eye without covering the cornea. Additionally, in some examples, the scleral flange, or other similar segment of the housing, can include a scleral lip or scleral seal about a perimeter of the portion of the device that interfaces with the eye. In some examples, the scleral lip or scleral seal can be shaped to facilitate retention of the active agent matrix to the housing, such as via friction fitting, nesting, clamping, or the like. In some examples, the scleral lip or scleral seal can additionally form a fluidic seal against the eye to minimize fluid transport across the scleral seal. In some examples, this can help concentrate delivery of the active agent to a specific region of the sclera and improve delivery of the active agent to the posterior segment of the eye.

In some examples, a pressure regulator can be operatively connected to the housing and adapted to induce negative pressure between the housing and the eye to couple the housing to the eye when in use. In some examples, the pressure regulator can form part of the housing, such as an integrated component of the housing or as part of a monolithic housing. In some examples, the pressure regulator can be a bulb, a pump, the like, or other suitable pressure regulator that can be operatively connected to the housing. The pressure regulator can generally be adapted to induce a negative pressure between the housing and the eye to couple the housing to the eye when in use. The negative pressure induced between the housing and the eye can be any pressure suitable to maintain the housing on the eye without significantly damaging the eye. In some examples, the pressure regulator can be adapted to induce a negative pressure of from about 0.98 atmospheres (atm) to about 0.1 atm between the housing and the eye. In yet other examples, the pressure regulator can be adapted to induce a negative pressure of from about 0.90 atm to about 0.3 atm. In still other examples, the pressure regulator can be adapted to induce a negative pressure of from about 0.8 atm to about 0.5 atm. In some examples, the pressure regulator can be adapted to reduce a pressure between the housing and the eye by an amount from about 0.1 atm to about 3 atm relative to atmospheric pressure. In yet other examples, the pressure regulator can be adapted to reduce a pressure between the housing and the eye by an amount from about 0.5 atm to about 1 atm relative to atmospheric pressure.

The active agent matrix can be coupled to the housing using any suitable coupling feature, such as an adhesive, stitching, friction-fitting, clips, clamps, magnets, snaps, hook and loop fasteners, the like, or combinations thereof. In some specific examples, the active agent matrix can be coupled to the housing via an adhesive. A variety of suitable adhesives can be used. Non-limiting examples can include a silicone adhesive, an acrylic adhesive, a polyurethane adhesive, the like, or combinations thereof. Further, the active agent matrix can generally be positioned to interface with the sclera of the eye, but not the cornea of the eye. In some examples, the active agent matrix can be formed of a plurality of segments that are positioned adjacent to one another to form an integral active agent matrix. In some specific examples, the active agent matrix can be formed from 2, 3, 4, or more individual segments positioned adjacent to one another. In some examples, the individual segments can be spaced apart from one another. In yet other examples, the individual segments can be positioned so that there is substantially no space between adjacent segments.

The active agent matrix can have a variety of suitable densities. In some specific examples, the active agent matrix can have a density of from about 0.15 grams/cubic centimeter (cc) to about 0.4 grams/cc prior to loading with the active agent composition. In yet other examples, the active agent matrix can have a density of from about 0.18 g/cc to about 0.35 g/cc prior to loading the active agent composition. In still other examples, the active agent matrix can have a density of from about 0.2 g/cc to about 0.31 g/cc prior to loading the active agent composition.

The active agent matrix can also have a variety of thicknesses. In some specific examples, the active agent matrix can have a thickness of from about 250 μm to about 600 μm prior to loading with the active agent composition. In yet other examples, the active agent matrix can have a thickness of from about 300 μm to about 500 μm prior to loading with the active agent composition. In still other examples, the active agent matrix can have a thickness of from about 350 μm to about 450 μm prior to loading with the active agent composition. The post-loading thickness of the active agent matrix can typically be greater than the pre-loading thickness of the active agent matrix. For example, in some cases, the post-loading thickness can be from about 2 times to about 6 times the pre-loading thickness. In yet other examples, the post-loading thickness can be from about 3 times to about 5 times the pre-loading thickness.

The active agent matrix can have a variety of ocular surface areas or ocular interface areas (i.e. the area of the active agent matrix that interfaces with the eye). In some examples, the ocular surface area of the active agent matrix can be from about 50 mm² to about 300 mm². In some additional examples, the ocular surface area of the active agent matrix can be from about 75 mm² to about 250 mm². In yet other examples, the ocular surface area of the active agent matrix can be from about 100 mm² to about 200 mm².

A method of treating an ophthalmic condition responsive to dexamethasone phosphate, or a salt thereof, in a subject is also described herein. Non-limiting examples of ophthalmic conditions that may be treatable with dexamethasone phosphate, or a salt thereof, include uveitis, age-related macular degeneration (AMD), diabetic retinopathy, diabetic macular edema, dry eye, post-operative inflammation, eye infection, allergic conjunctivitis, corneal trauma, infiltrative keratitis, staphylococcal marginal keratitis, posterior blepharitis, ocular herpetic disease, cystoid macular edema (CME), diabetic retinopathy, Behçet's disease, ocular pain, or a combination thereof.

In some specific examples, the administration of dexamethasone phosphate, or a salt thereof, can be performed via passive administration. As such, dexamethasone phosphate, or a salt thereof, can be topically administered to the eye and allowed to passively diffuse into the eye. In some examples, passive administration can employ penetration enhancers or other suitable delivery aids to increase the rate at which dexamethasone phosphate, or a salt thereof, is delivered to the eye. In other examples, passive administration does not employ penetration enhancers or the like. In some specific examples, passive administration can be non-invasive passive administration.

In some examples, the administration of dexamethasone phosphate, or a salt thereof, can be performed via active administration. Active administration can employ iontophoresis, electroporation, ultrasound, microneedles, the like, or a combination thereof to actively deliver dexamethasone phosphate, or a salt thereof, to the eye. However, it is noted that where active administration is non-invasive, microneedles or the other administration methods that are configured to pierce or puncture an ocular surface are not considered non-invasive administration techniques. As drug delivery methods employing iontophoresis, electroporation, ultrasound, or microneedles are generally known in the art, such methods will not be discussed in detail. However, it is to be understood that such methods, and other similar methods, are considered within the scope of the present disclosure. In some specific examples, active administration can include iontophoretic administration of dexamethasone phosphate, or a salt thereof, to the eye. In yet other examples, active administration can include electroporation or electroporation-facilitated delivery of dexamethasone phosphate, or a salt thereof, to the eye. In some examples, active administration can include ultrasound or ultrasound-facilitated delivery of dexamethasone phosphate, or a salt thereof, to the eye. In some examples, active administration can employ microneedles to facilitate delivery of dexamethasone phosphate, or a salt thereof, to the eye.

Whatever the mode of administration, dexamethasone phosphate, or a salt thereof, can typically be administered via a therapeutically effective dosing regimen that includes one or more continuous administration periods. More specifically, each administration event is typically performed for a continuous or consecutive period. Generally, the continuous period is less than one week. In some additional examples, the continuous period is less than or equal to 5 days, less than or equal to 3 days, or less than or equal to 1 day (i.e. 24 hours). In some specific examples, the consecutive period can be a period of from about 1 minute to about 30 minutes. In yet other examples, the consecutive period can be a period of from about 2 minutes to about 20 minutes, from about 3 minutes to about 15 minutes, from about 4 minutes to about 10 minutes, or from about 5 minutes to about 8 minutes. It is noted that the continuous or consecutive period can be adjusted based on the concentration of the dexamethasone phosphate, or a salt thereof. For example, where a longer administration event or administration period is desired, a lower concentration of dexamethasone phosphate, or a salt thereof, can be used. Conversely, where a shorter administration event or administration period is desired, a greater concentration of dexamethasone phosphate, or a salt thereof, can be used.

Thus, each administration event can be a sufficient continuous period of time to introduce a threshold dose of dexamethasone phosphate, or a salt thereof, to the eye. In some examples, the threshold dose can be considerably higher than an amount administered via an eye drop. In some cases, the threshold dose can deliver at least about 5 times more dexamethasone phosphate, or a salt thereof, to the eye than an eye drop. In some cases, the threshold dose can depend on the type and severity of the condition being treated, the specific individual being treated, etc. In some examples, the threshold dose can be an amount from about 0.1 mg to about 30 mg of dexamethasone phosphate, or a salt thereof. In yet other examples, the threshold dose can be an amount from about 0.2 mg to about 10 mg of dexamethasone phosphate, or a salt thereof. In still other examples, the threshold dose can be an amount from about 0.5 mg to about 5 mg of dexamethasone phosphate, or a salt thereof. In some specific examples, the threshold dose can be an amount from about 0.1 mg to about 0.5 mg of dexamethasone phosphate, or a salt thereof. In other specific examples, the threshold dose can be an amount from about 0.2 mg to about 1 mg, about 0.3 mg to about 2 mg, or about 0.25 mg to about 1.5 mg of dexamethasone phosphate, or a salt thereof. In yet other specific examples, the threshold dose can be an amount from about 1 mg to about 8 mg, about 0.5 mg to about 4 mg, about 5 mg to about 12 mg, about 6 mg to about 16 mg, or about 10 mg to about 20 mg.

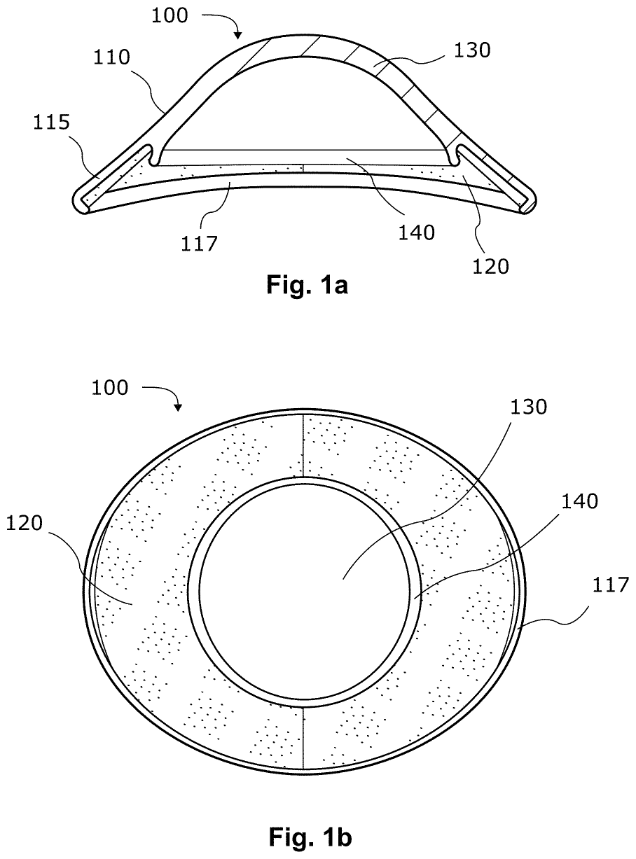

Turning now to the figures, FIGS. 1a and 1b illustrate one example of a non-invasive ocular drug delivery device 100 having a housing 110 and an active agent matrix 120 coupled thereto. In this particular example, the active agent matrix 120 includes two semicircle segments, but can include a single segment or other suitable number of segments. The housing 100 includes a corneal dome 130 shaped to cover a cornea of an eye. Additionally, the housing includes a corneal seal 140 positioned about a perimeter of the corneal dome 130 to form a fluidic seal against the eye when in use to minimize fluid transport into the corneal dome 130. The housing also includes a scleral flange 115 positioned to cover a portion of the sclera of an eye without covering the cornea. A scleral lip or scleral seal 117 is disposed about a perimeter of the scleral flange 115.

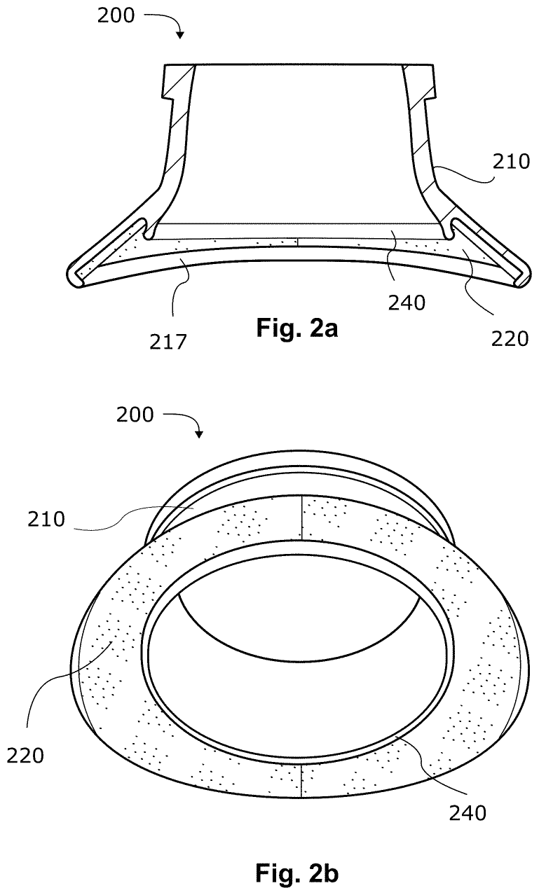

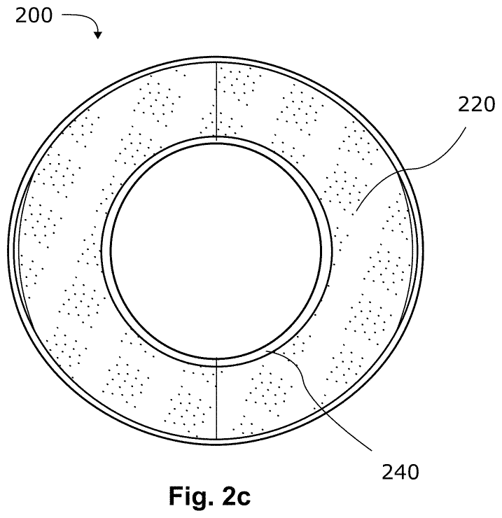

FIGS. 2a, 2b, and 2c illustrate an alternative example of a non-invasive ocular drug delivery device 200 having a housing 210 and an active agent matrix 220 coupled thereto. In this particular example, the housing 200 does not include a corneal dome. As such, the cornea of the eye can be exposed to ambient conditions during use of this particular example of the device 200. Nonetheless, the device 200 still includes a corneal seal 240 to minimize fluid transport across the surface of the eye to the cornea. This can minimize surface contact of the active agent with the sensitive cornea. The device 200 can also include a scleral lip or scleral seal 217 adapted to contain topical delivery of the active agent between the corneal seal 240 and the scleral seal 217.

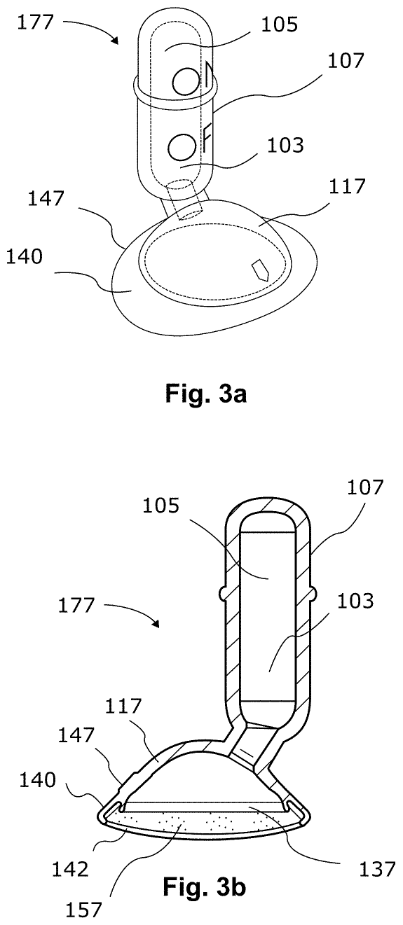

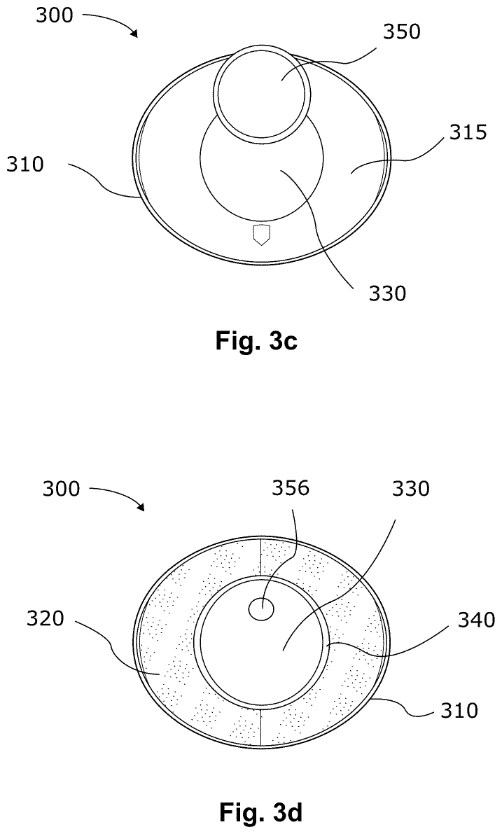

FIGS. 3a, 3b, 3c, and 3d illustrate yet another example of a non-invasive ocular delivery drug device 300. In this example, the device 300 includes a housing 310 with an active agent matrix 320 coupled thereto. Additionally, a pressure regulator 350 is coupled to a corneal dome 330 of the housing via pressure channel 356 to induce negative pressure between the housing and the eye. In this particular example, the negative pressure can be isolated to the corneal region of the device because the device includes a corneal dome 330 and a corneal seal 340 to maintain the pressure within the corneal region of the device. Additionally, in this particular example, the pressure regulator 350 can be marked, or include instructions, for applying device 300 to the eye and removing the device 300 from the eye. For example, segment 352 of the pressure regulator 350 can be marked for placement of device 300 on the eye, whereas segment 354 can be marked for removal of device 300 from the eye. In some examples, the segment 352 can form a lesser volume of the pressure regulator 350 than segment 354. As such, depressing segment 352 prior to application of the device 300 to the eye can generate sufficient negative pressure between the eye and the device 300 to couple the device 300 to the eye when segment 352 is released. Conversely, segment 354 can form a greater volume of the pressure regulator 350 than segment 352. As such, when it is desirable to remove the device 300 from the eye, depression of segment 354 can induce sufficient positive pressure between the device 300 and the eye to facilitate removal of the device 300 from the eye.

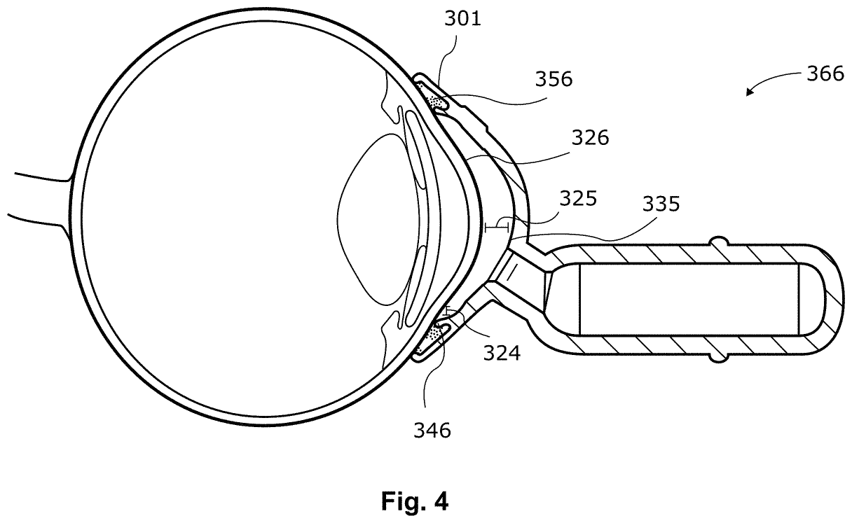

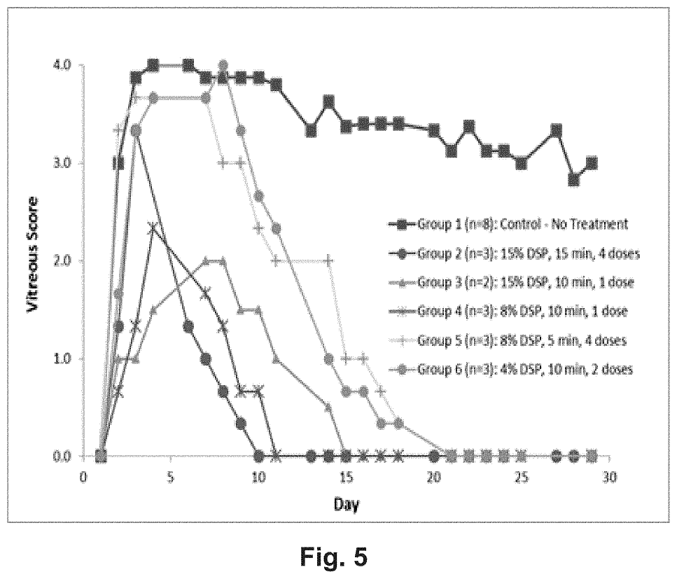

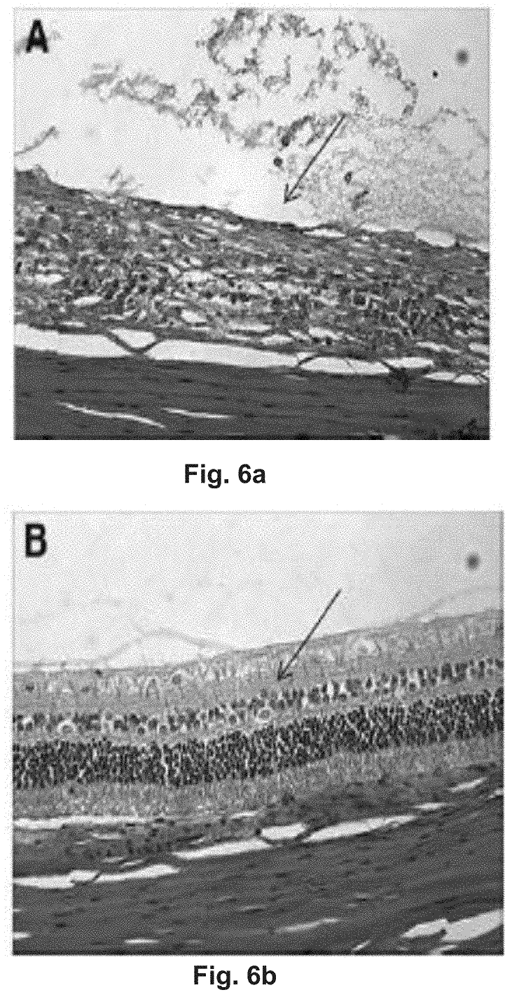

FIG. 4 illustrates an example of the device 300 coupled to an eye. As can be seen in this particular figure, a gap 362 can be maintained between an inner surface 332 of the housing and the cornea 360 so as to minimize contact of the housing 310 with the cornea 360. Additionally, a distance 364 can be maintained between the perimeter of the cornea 360 and the corneal seal 340 so as to maintain a fluidic seal about the cornea and minimize fluid transport across the surface of the eye to the cornea 360.

### EXAMPLES

### Example 1—Treatment of Experimental Uveitis in Rabbits

Dexamethasone sodium phosphate (DSP) USP grade was obtained from Letco Products (Decatur, Ala.). The concentrations of DSP solution were 4.0%, 8.0%, and 15.0% w/v. All DSP formulations contained 0.01% w/v of EDTA (Sigma-Aldrich, St. Louis, Mo.) with pH adjusted to 7.0 with 1M hydrochloric acid (LabChem, Zelienople, Pa.) and were freshly prepared in doubly deionized water on the day of dosing using an aseptic technique. The applicator for use in rabbit studies was fabricated from medical grade silicone rubber, which incorporated a customized active agent matrix (3-5 mm wide). Young adult New Zealand White rabbits (both male and female), each weighing 3-4 kg, were obtained from Western Oregon Rabbit Co. (Philomath, Oreg.). This study complied with the ARVO Statement for the use of Animals in Ophthalmic and Vision Research and was approved by The University of Utah Institutional Animal Care and Use Committee (Salt Lake City, Utah). All animals were acclimated and observed for health issues for at least 2 weeks before being used in the study. Freund's complete adjuvant (FCA) and Mycobacterium tuberculosis H37Ra antigen were purchased from Difco Laboratories, Inc. (Detroit, Mich.), ketamine hydrochloride injectable USP (100 mg/mL), and sodium chloride 0.9% USP were from Hospira, Inc. (Lake Forest, Ill.). Proparacaine hydrochloride ophthalmic solution and gentamicin sulfate ophthalmic solution were from Bausch & Lomb (Tampa, Fla.). Cyclopentolate hydrochloride ophthalmic solution was from Alcon Laboratories (Fort Worth, Tex.). The binocular indirect ophthalmoscope used was the Keeler All Pupil II from Keeler Instruments (Broomall, Pa.) and it was complemented with the double aspheric lens 20 D/50 mm for posterior chamber examination from Volk Optical, Inc. (Mentor, Ohio).

Twenty-three animals were randomly assigned into 6 groups according to Table 1 after uveitis induction of the right eye. Left eyes were not induced with uveitis to provide some vision in the animals throughout the study. The DSP treatment was on the affected eye (right eye). The first dose occurred ˜30 minutes after the uveitis induction on Day 1. Ocular examinations and clinical observation were performed during the weekday before and after each dosing. Following the final observations on Day 29, animals were anesthetized with a 2.5 mL intramuscular injection containing 5 mg ketamine and 30 mg xylazine per mL. Depth of anesthesia was confirmed by absence of corneal blink reflex or toe pinch response to ensure humane euthanasia. The animal was then sacrificed by an intracardiac injection of 2 mL of saturated KCl with a 3 mL syringe and 18 GA×1″ needle. The eyes were collected and processed for histological evaluation. The severity of the uveitic conditions limited the number of rabbits per group to 3 in the first part of the study. With the successful experience of the first part of the study, the same number of animals per group was kept for the rest of the study. The study was conducted in 3 parts, and each time a control group was evaluated with the treatment group(s). Then, the results were pooled for analysis.

TABLE 1
Study Design

| | Number of | DSP Concentration | Application | Day of |
|---|---|---|---|---|
| Group | Animals | (w/v %) | Time (Minutes) | Dosing |
| 1 | 8 | No Treatment | n/a | n/a |
| 2 | 3 | 15 | 15 | 1, 8, 15, 22 |
| 3 | 3 | 15 | 10 | 1 |
| 4 | 3 | 8 | 10 | 1 |
| 5 | 3 | 8 | 5 | 1, 8, 15, 22 |
| 6 | 3 | 4 | 10 | 1, 8 |

For uveitis induction, rabbits were preimmunized by subcutaneous injections of 0.5 mL FCA H37Ra, a suspension of Mycobacterium tuberculosis H37Ra antigen in FCA. The Freund's Complete Adjuvant H37Ra containing 20 mg/mL of antigen was prepared by mixing dried M. tuberculosis H37Ra antigen with the FCA. The preimmunized injections were in the dorsal area of the animal's neck and occurred at 19 and 12 days before induction of uveitis. Then uveitis was induced on Day 1 by 100 mL IVT injection of a suspension containing 33 mg of the M. tuberculosis H37Ra antigen in sterile balanced salt solution on the right eye using Hamilton syringe with a 30 Ga×½ needle. No uveitis induction was performed on the left eye. Although a second IVT induction was planned on Day 15, it was not given due to the severity of inflammation in the control group eyes (Group 1). Rabbits were anesthetized with a 2.5 mL intramuscular injection containing 5 mg ketamine and 30 mg xylazine per mL. One drop each of proparacaine and gentamicin was administered to the eye before the IVT injection. The IVT injection entered through the limbus in the superior portion of the sclera and administered approximately in the middle of the vitreous.

Each rabbit was placed in a rabbit restrainer to limit movement during the DSP administration. One drop of sterile proparacaine hydrochloride ophthalmic solution, a local anesthetic, was given to the right eye of each rabbit ˜5 min before dose administration. DSP solution (250 μL) was loaded into the applicator using an Eppendorf pipettor. The drug solution saturated the carrier matrix uniformly within a minute. Then, the applicator containing the drug formulation was gently applied to the scleral surface of the right eye of each rabbit. The position of the applicator was checked to ensure that the drug matrix was in immediate contact with the white scleral part of the eye, but not the cornea. Digital laboratory timers were used for accurate application times (treatment duration) of 5, 10, or 15 min. After the given treatment duration, the applicator was carefully removed from the eye.

Body weights of the animals were taken upon arrival, immediately after EAU induction, and before sacrifice. All eyes of the animals (both left and right eyes) were examined by indirect ophthalmoscopy to evaluate respective effects on the cornea, conjunctiva, anterior chamber (AC), vitreous, posterior chamber, and sclera. One to 2 drops each of phenylephrine hydrochloride ophthalmic solution and cyclopentolate hydrochloride ophthalmic solution was used as a mydriatic. Observations pertaining to conjunctival injection, chemosis, discharge, and clarity of anterior and posterior segment of the eye were made, scored, and recorded. An average of all scores over the course of study was calculated for comparison. A modified McDonald-Shadduck scale was used for grading inflammation.

The enucleated eyes were stored in Davidson's solution (i.e. 34.7% deionized water, 11.1% glacial acetic acid, 32.0% ethanol, and 22.2% formalin) for 24 hrs, and then transferred to plastic conical tubes containing 20 mL of 70% ethanol in water. The eyes were sent for histopathological processing and evaluation at Colorado Histo-Prep (Fort Collins, Colo.). A central cut of the eye globe was taken as well as 2 cuts on either side of the central cut (calottes) at trim. For each eye, the central cut was placed into one cassette and the 2 calottes were placed together into a separate cassette. The tissues were processed, embedded in paraffin wax, sectioned by microtomy, and stained. Histopathology of the tissues was conducted on slides stained with hematoxylin and eosin. The pathologist who evaluated the tissues had no prior knowledge of the specific pharmacologic activity or formulation of the test articles. Standardized toxicologic pathology criteria and nomenclature for the rabbit were used to categorize microscopic tissue changes. For anterior section, the conjunctiva, cornea, AC, trabecular meshwork, iris, and ciliary body were evaluated and scored from 0 (normal) to 4 (marked) for signs of inflammation, including edema/congestion of the conjunctiva, ciliary body, cornea, inflammatory cell infiltration in the conjunctiva, cornea, AC, trabecular meshwork, iris, ciliary body, and neovascularization on the cornea. Scores from each tissue were combined to give a total inflammatory score of anterior section (maximum score=40). For posterior section, the vitreous, choroid, and retina were also scored from 0 (normal) to 4 (marked) for signs of inflammatory cell infiltration.

All scores are reported as mean—standard deviation (unless otherwise indicated). The differences in mean score between the control group and each DSP treatment group were evaluated by the Wilcoxon rank-sum test. This included vitreous score, AC score, and conjunctiva injection score from clinical observation, and inflammatory score and inflammatory cell infiltration score from histopathological examination. Differences were considered significant at P<0.05.

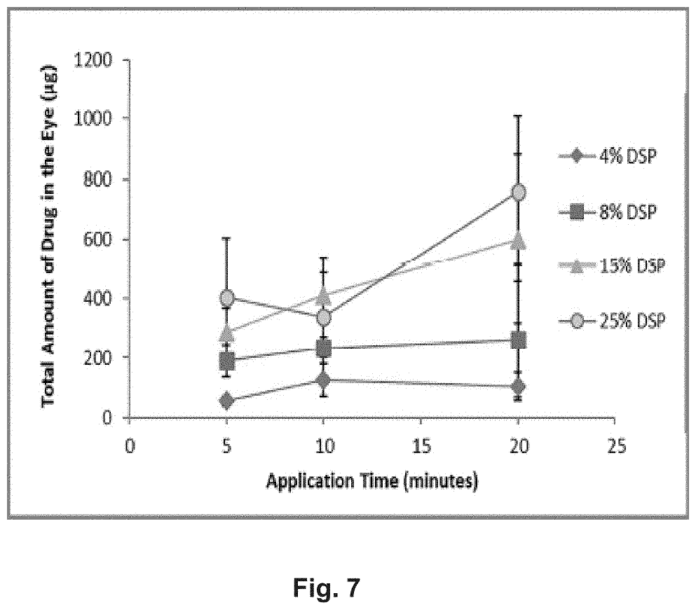

All right eyes showed signs of inflammation within a day after the induction. Left eyes showed no signs of inflammation through the end of the study. One rabbit in Group 3 died due to an unknown cause during the preimmunization period and before the initiation of DSP dosing. Inflammation occurred more significantly in the posterior chamber than in the AC. All treatment regimens reduced the signs of uveitis. However, the most prominent finding from ophthalmic examination in assessing the severity of uveitis is the vitreous opacity (FIG. 7). The observations from each section of the eye are as follows:

Vitreous. All animals in the control group (Group 1) reached a severe uveitic state (i.e. scores of 3 or 4 for the vitreous), which remained on average above a score of 3 throughout the 28 days of study. Vitreous opacity increased steadily for the first 4 days after initiation of uveitis in all 5 groups. The opacity in Group 1 (control) increased the most. Scores for Group 1 animals decreased slightly around Day 13, but remained on average above a score of 3 throughout the experiment. By Day 4, Groups 2, 3, and 4 had reached the highest scores they would attain and began to decrease steadily thereafter. Group 5 scores began a steady decrease on Day 8, while those for Group 6 began to decrease on Day 10. There were clear decreases in vitreous opacity scores in all treatment groups, while the control group scores remained high. Group 2 animals showed a steady decrease in vitreous opacity scores until reaching zero on Day 10 and remaining at zero throughout the remainder of the study. Group 3 (15% DSP, 10 min, 1 dose) reached zero on Day 15, Group 5 (8% DSP, 10 min, 4 doses) on Day 11, and Groups 4 (8% DSP, 5 min, 1 dose) and 6 (4% DSP, 10 min, 2 doses) reached 0 on Days 21 and 22, respectively. Averaged vitreous scores over the course of study are presented in Table 3. Over the course of study, the average score of vitreous for the control group was 3.3-1.1 and all the DSP treatment groups (Groups 2-6) were statistically significantly lower than the control.

TABLE 3
Inflammation Scores from Clinical Observations
using Indirect Ophthalmoscope

| | Inflammation Score | | |
|---|---|---|---|
| Treatment | Conjunctival | Anterior | |
| Regimen | Injection | Chamber | Vitreous |
| Group 1 | 0.9 ± 0.8 | 0.5 ± 0.5 | 3.3 ± 1.1 |
| Group 2 | 0.5 ± 0.4 | 0.1 ± 0.2 | 0.4 ± 1.0 |
| Group 3 | 0.3 ± 0.4 | 0.1 ± 0.2 | 0.6 ± 1.2 |
| Group 4 | 0.5 ± 0.5 | 0.3 ± 0.4 | 0.4 ± 0.8 |
| Group 5 | 0.9 ± 0.8 | 0.5 ± 0.5 | 1.4 ± 1.7 |
| | (P = 0.3) | (P = 0.6) | |
| Group 6 | 0.5 ± 0.5 | 0.4 ± 0.6 | 1.4 ± 1.6 |
| | | (P = 0.1) | |

Anterior chamber. No hypopyon, synechia, or flare was noted in this study. Some fibrin formation in the AC was observed in all groups with slightly different degrees. The signs of inflammation in the AC were not drastic even with the control group. Average AC scores over the course of study was less than 1.0 for all groups (Table 3). The trends of the AC scores were similar for Group 1, Group 5, and Group 6. The average daily score of Group 5 was equal to that of the control group. Group 6 also had fibrin present throughout the study with an average daily score slightly lower than the controls, but not statistically significant. Group 4 (8%, 10 min, single dose) displayed a low fibrin score over the course of the study with an average of 0.3, which is significantly lower than the average of 0.5 for the control group (Group 1). Group 2 (15%, 15 min, 4 weekly doses) and Group 3 (15%, 10 min, single dose) reached an AC score of 0 within about 1 week after the first treatment. Both groups showed the averaged AC score of 0.1, which is significantly lower than the control group.

Conjunctival injection. Mild to moderate conjunctival injection was present in all animals and was observed throughout the study. Averaged group scores over the course of treatment are presented in Table 3. All treatment groups except Group 5 showed slightly lower average conjunctiva scores over the course of study than the control group (Group 1). The average conjunctiva scores of Group 5 were equal to the control group. There were day to day variations as well as an overall downward trend over the entire experiment in all groups (i.e. the average score ranged from 0 to 3 in the first 2 weeks and from 0 to 1 in the last 2 weeks). In Group 1, conjunctival injection declined slowly over the course of the experiment, but was still present until the end. Some irritation from placement of the DSP was observed in the DSP treatment groups. In Groups 2, 5, and 6 (multiple doses), slight increases were observed after each application followed by improvement until the next application. Conjunctival injection scores in Groups 3 and 4 (single dose) declined after Day 3, were minimal after about 10 days, and completely resolved by Day 22.

Chemosis. Mild chemosis was found in all groups. Overall chemosis was minor, with no group having an average chemosis score greater than 1 at any point. In Group 1 animals (controls), chemosis decreased slowly, although with variation, throughout the study. Chemosis increased slightly after DSP treatment, a trend similar to that seen with conjunctival injection. Groups 2 and 5 showed mild chemosis immediately after each dosing, but resolving to 0 generally within a day. Groups 4 and 6 showed some variations in chemosis scores and reached 0 after Day 11, with Group 6 showing a slight reoccurrence on Days 16 through 18. Neither Group 3 rabbits displayed any significant chemosis.

Conjunctival discharge. Discharge was noted in all groups in a random manner. Discharge never exceeded a score of 1. There was an undistinguishable trend between the treatment regimens and the control.

Cornea. A low grade of cornea cloudiness, mostly with scores of <1, was found in some rabbits in all groups (untreated control group and treatment groups). The corneal haze observed in all rabbits faded with time. Overall, the incidence and severity of corneal haze in treatment groups appeared to be lower than the control group.

Body weight. Group 1 animals (controls) maintained their average body weight throughout the study. Group 2, with the highest dosing of DSP (4 weekly doses of 15% for 15 min), had an average loss of body weight of 0.3 kg, or about 8%. There were no significant weight changes in any of the other treatment groups.

Histopathology of uveitis eyes—The eyes were collected at the end of the study on Day 29 for histopathology evaluation. The average inflammation scores for both anterior and posterior sections of the eyes graded by a veterinarian pathologist are presented in Table 4.

TABLE 4
Inflammation Scores and Inflammatory Cell Infiltration
Score from Histopathology Examination

| | Total Inflammatory | Inflammatory Cell Infiltration Score | |
|---|---|---|---|
| Treatment | Score of Anterior | Anterior | Posterior |
| Regimen | Section | Section | Section |
| Group 1 | 4.4 ± 2.6 | 0.7 ± 1.0 | 2.9 ± 1.2 |
| Group 2 | 0.2 ± 0.4 | 0.0 ± 0.2 | 0.1 ± 0.3 |
| Group 3 | 1.0 ± 1.1 | 0.2 ± 0.4 | 1.8 ± 1.5 |
| Group 4 | 1.8 ± 0.7 | 0.3 ± 0.7 | 1.2 ± 0.9 |
| Group 5 | 1.4 ± 1.7 | 0.2 ± 0.8 | 1.9 ± 1.6 |
| Group 6 | 1.9 ± 1.1 | 0.3 ± 0.7 | 2.9 ± 1.0 |
| | | | (P = 0.7) |

Anterior section. No edema or congestion of conjunctiva, ciliary body, or cornea was observed in all groups. No neovascularization on the cornea was found in this study. The total inflammatory score of anterior section was 4.4 on average for the untreated eye, whereas the DSP treatment groups were significantly lower. The efficacy of DSP treatment in the anterior section appears to be related to DSP concentrations. Groups 2 and 3, where the DSP concentration was 15%, the averaged total inflammatory scores were 0.2 and 1.0, respectively; Groups 4 and 5, where the DSP concentration was 8%, the total scores were 1.8 and 1.4, respectively; and Group 6, where the DSP concentration was the lowest at 4%, the total score was the highest among treatment groups at 1.9. Similarly, the inflammatory cell infiltrations into the anterior section of the eye were less in all DSP treatment groups compared to the control. This was reflected by the lower of inflammatory cell infiltration scores of the treatment groups compared to the control group. However, there was no obvious efficacy-concentration relationship among the treatment groups. All animals in Group 1 (untreated) had inflammatory cell infiltrations to the conjunctiva, cornea, AC, trabecular meshwork, iris, and/or ciliary body with the average inflammatory cell infiltration score of 0.7 for the whole anterior section. In contrast, the average inflammatory cell infiltration score of Group 2 (15% DSP, 15 min, 4 doses) was 0.0. No cell infiltrations in the conjunctiva, AC, trabecular meshwork, iris, or ciliary body were found in this group. For Group 3 (15% DSP, 15 min, 1 dose), Group 4 (8% DSP, 10 min, 1 dose), Group 5 (8% DSP, 5 min, 4 doses), and Group 6 (4% DSP, 10 min, 2 doses), few inflammatory cell infiltrations were found in ciliary body, conjunctiva, and/or cornea tissues, but not in the other anterior tissues (i.e., AC, trabecular meshwork, and iris) with the average inflammatory cell infiltration scores of 0.2, 0.3, 0.2, and 0.3, respectively.

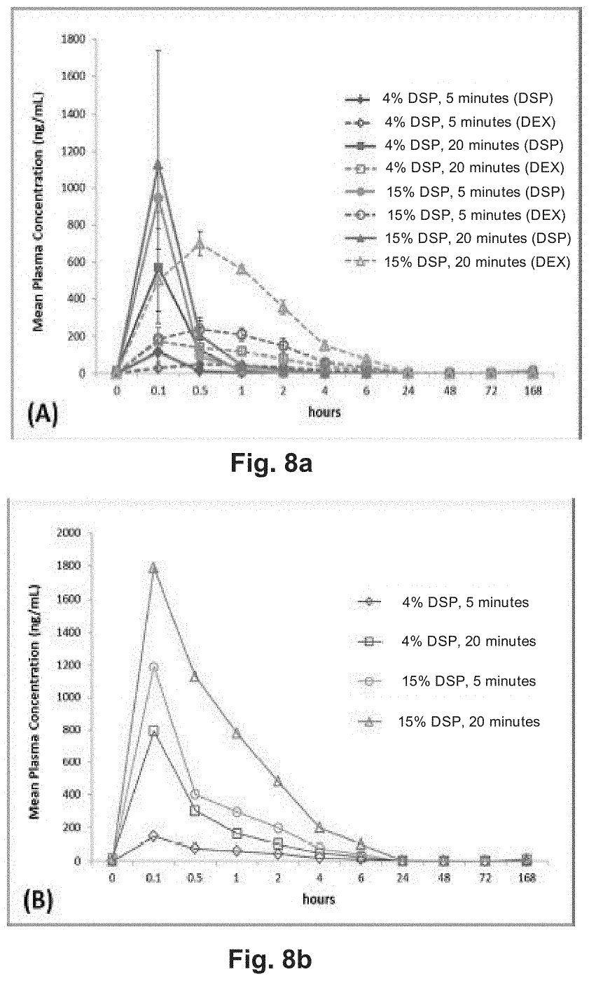

Posterior section. The overall inflammatory cell infiltration scores of the posterior section calculated from the respective individual vitreous, choroid, and retina scores are summarized in Table 4. The results show that all DSP treatment groups, except the lowest dosing group (Group 6), were less inflamed in the posterior section than the controls (Group 1). The untreated animals showed moderate to severe inflammation in respective vitreous, choroid, and retina tissues with the average inflammatory cell infiltration score of 2.9. This indicates that intermediate and posterior uveitis were persistent in the control group for 29 days, consistent with the clinical observations. Group 2 animals had almost no pathological signs of uveitis present, with the average inflammatory cell infiltration score of 0.1. This supports that such eyes made a full recovery from induced intermediate and posterior uveitis. The differences in the photoreceptor layer appearance between the untreated eye (Group 1) and the eye from the highest dose regimen (Group 2) can be seen in FIGS. 8a and 8b. The posterior tissues of the treated eye appeared to be healthy with minimal inflammation, where it appeared to be completely impaired in the untreated eye. Histopathology of Group 3 (15% DSP, 10 min, 1 dose), Group 4 (8% DSP, 10 min, 1 dose), and Group 5 (8% DSP, 5 min, 4 doses) showed minimal to mild inflammation with the average infiltration scores of 1.8, 1.2, and 1.9, respectively. All animals in the lowest dosing group (Group 6) had posterior section inflammation nearly identical to the control group.

### Example 2—Ocular Drug Distribution and Safety of Dexamethasone Sodium Phosphate

Dexamethasone sodium phosphate (DSP) USP grade was supplied from Letco Products (Decatur, Ala.). The concentrations of DSP solution were 4.0%, 8.0%, 15.0%, and 25.0% w/v. All DSP solutions containing 0.01% w/v of EDTA (Sigma-Aldrich, St. Louis, Mo.) with the pH adjusted to 7.0 using 1.0 M hydrochloric acid (LabChem, Zelienople, Pa.) were freshly prepared in double deionized water on the day of dosing using an aseptic technique. The non-invasive ocular drug delivery device for use in this study was fabricated from medical grade silicone rubber and a proprietary sponge material. Ketamine hydrochloride injectable USP (100 mg/mL) and sodium chloride 0.9% USP were from Hospira, Inc. (Lake Forest, Ill.); proparacaine hydrochloride ophthalmic solution was from Bausch & Lomb (Tampa, Fla.); cyclopentolate hydrochloride ophthalmic solution was from Alcon Laboratories (Fort Worth, Tex.); xyrazine and potassium chloride (KCl) were from Sigma-Aldrich (St. Louis, Mo.). Syringes and needles were from Becton, Dickinson and Company (Franklin Lakes, N.J.). The binocular indirect ophthalmoscope used was the Keeler All Pupil II from Keeler Instruments (Broomall, Pa.) and it was complemented with the double aspheric lens 20 D/50 mm for posterior chamber examination from Volk Optical Inc (Mentor, Ohio). Young adult New Zealand White rabbits each weighing 3-4 kg were obtained from Western Oregon Rabbit Co. (Philomath, Oreg.). This study complied with the ARVO Statement for the use of Animals in Ophthalmic and Vision Research and was approved by The University of Utah Institutional Animal Care and Use Committee (Salt Lake City, Utah). All animals were acclimated and observed for health issues for at least two weeks prior to being used in the study.

Sixty animals were randomly assigned into twenty groups of three (n=3) for three main studies: ocular drug distribution, ocular toxicity, and toxicokinetics.

For the ocular drug distribution study, there were a total of twelve groups. The test parameters included four DSP concentrations (i.e., 4%, 8%, 15%, and 25% w/v) and three application times (i.e., 5, 10, and 20 minutes). Each group received a single DSP treatment via the non-invasive ocular drug delivery device at a pre-specified concentration and application time on both eyes concurrently (within 10-20 seconds apart). The rabbits were sacrificed immediately after dosing (generally within 5 minutes). The eyes were then enucleated and analyzed for DSP and DEX using HPLC. A total of 6 eyes were used for averaging the amount of the drug in each group. The rationale for this study was to answer whether or not a single application of the non-invasive ocular drug delivery device can deliver a meaningful amount of DSP into the deeper eye tissues. Since there is no established minimum effective concentration of DEX or DSP in ocular tissues, the target concentration of DSP in each eye tissue (immediately after the application) that is considered meaningful was arbitrarily set at 1 μg/g. This was based on the fact that 1 μg/mL DEX was the quantification limit of the HPLC assay in this study. This number can very well be on the high side as even a concentration of DEX at 10⁻⁷M (˜40 ng/mL) can inhibit prostaglandin release from rabbit coronary microvessel endothelium.

For the ocular toxicity study, there were four groups. The longest application time of interest, 20 minutes, was selected for testing safety and tolerability of the four DSP concentrations. Each rabbit received a weekly DSP administration via application of the non-invasive ocular drug delivery device (i.e., 4%, 8%, 15%, or 25% DSP concentrations) for 20 minutes in one eye (right eye) leaving the other (left eye) as an untreated control. The total exposure was 12 doses over the period of 12 weeks. Clinical observations were performed on weekdays, and before and after each dosing. Following the final observations (i.e., one week after the last dose), the rabbits were sacrificed and the eyes were processed for histological evaluation.

For the toxicokinetic study, there were four groups of rabbit. Each group received a single dose of 5 or 20 minute application of 4% or 15% of DSP in one eye. Blood was collected and processed for plasma at predose, 5, 30, 60, 120, 240, and 360 minutes, and 24, 48, 72, 96, and 168 hours after administration. Plasma concentration analysis for DEX and DSP was performed using LC-MS.

At the termination point in all three studies, the animals received a 2.5 mL intramuscular injection containing 5 mg ketamine and 30 mg xylazine per mL as general anesthetic. For each animal, the depth of anesthesia was confirmed by absence of corneal blink reflex or toe pinch response to ensure humane euthanasia. The animal was then sacrificed by an intracardiac injection of 2 mL of saturated KCl with a 3 mL syringe and 18 GAλ 1″ needle. The eyes were collected and processed for drug analysis or histological evaluation.

Each rabbit was placed in a rabbit restrainer to limit movement during administration of DSP via the non-invasive ocular drug delivery device. One drop of sterile proparacaine hydrochloride (a local anesthetic) was put on the eye (to be treated) 5 minutes before dose administration. DSP solution (250 μl) was loaded onto the annular active agent matrix of the non-invasive ocular drug delivery device using an Eppendorf pipettor. Then, the non-invasive ocular drug delivery device containing the DSP solution was gently applied to the scleral surface of the eye of each rabbit. The position of the device was checked to ensure that the active agent matrix was in immediate contact with the white part of the eye but not the cornea. Digital timers were used for accurate application times (i.e., 5, 10, or 20 minutes). After the given application duration, the applicator was carefully removed from the eye.

For drug analysis, the eyes were dissected into seven tissue sections: anterior chamber, lens, retina-choroid, cornea, vitreous, conjunctiva, and sclera. The anterior chamber consists of iris, ciliary muscles, and aqueous humor. After dissection, the drug was extracted from each tissue overnight with 5 mL of the extraction solvent (60% chloroform-40% methanol). The tissue was then separated from the extraction solution by centrifuge at 3400 rpm for 10 minutes. The extraction solutions were concentrated by evaporation of the solvent in a water bath at 50° C., using nitrogen gas, and then reconstituted in 1 mL of the reconstitution solvent (95% methanol/5% 1M HCl). The amounts of total DSP and DEX in the eye tissues were then determined by HPLC analysis.

For histopathology, the enucleated eyes were stored in Davidson's solution (i.e., 34.7% deionized water, 11.1% glacial acetic acid, 32.0% ethanol, and 22.2% formalin) for 24 hours and then transferred to plastic conical tubes containing 20 mL of 70% ethanol in water. The eyes were sent for histopathological processing and evaluation at Colorado Histo-Prep (Fort Collins, Colo.).

Blood was collected at predose (−20 minutes), 5, 30, 60, 120, 240, and 360 minutes, and 24, 48, 72, and 168 hours after DSP application via the non-invasive ocular drug delivery device. Approximately 1 mL of blood was collected by direct venipuncture of the jugular vein with a 3 mL syringe and 21 GA×1″ needle. Blood was immediately transferred into anticoagulant (potassium EDTA) coated microcentrifuge tubes. Blood was then centrifuged for five minutes at 3000×G at 4° C. Plasma was immediately separated into another microcentrifuge tube then kept in −20° C. freezer for LC-MS analysis.

The amounts of DSP and DEX in the eye tissues were determined by HPLC analysis. The HPLC system used was Waters 2695 separation module equipped with Waters 2487 dual wavelength detector (Waters Corporation, Milford, Mass.) and Kinetex C18 column 2.6 μm 100×4.6 mm (Phenomenex, Torrance, Calif.). All the chemical reagents for making HPLC mobile phases were HPLC grade from Sigma-Aldrich (St. Louis, Mo.). The mobile phase was 30% by volume of acetonitrile and 0.1% by volume of trifluoroacetic acid (99%) in distilled deionized water. The HPLC method was isocratic with a 1.2 mL/min flow rate and column temperature was 30° C. The injection volume was 10 μL. A single UV wavelength mode was set at λ=240 nm. Retention times for DSP and DEX were 4.2 and 6.9 min, respectively. The DSP and DEX standard curves of 0.0005 to 0.5 mg/mL (i.e., concentration vs. absorbance) were generated. The lower limit of quantification of this method was 0.001 mg/mL.

All of the plasma analyses for DSP and DEX were performed at Tandem Labs (Salt Lake City, Utah) using LCMS. Briefly, the samples were assayed by Shimadzu SCL-10A controller with LC-10AD pump. The mobile phase was 50% by volume of 10 mM ammonium acetate and 50% by volume of methanol. The HPLC column was a XBridge Phenyl column, 5 μm, 50×2.0 mm. An isocratic elution was applied at 0.500 mL/min flow rate and column temperature was 30° C. An API 5000 (Applied Biosystem/Sciex) mass detector with an electrospray interface in positive mode (source temperature set at 400° C.) was used to detect the MS/MS transition m/z 393 to m/z 373.4 for DEX and m/z 473 to m/z 435 for DSP. The injection volume was 10 μL. The retention times for DSP and DEX were 1.2 and 2.5 min, respectively. DSP and Dex standard curves of 0.2 to 200 ng/mL were generated. The limit of quantitation (LOQ) of this method was 1 ng/mL.

Toxicokinetic data analysis was based on standard noncompartmental pharmacokinetic methods. Plasma concentration of DSP equivalent was used in the analysis to express systemic exposure of DSP and DEX as a single entity. The DSP equivalent was calculated by converting DEX to DSP using 392.5 g of DEX equivalent to 516.4 g of DSP. The maximum observed plasma concentration (Cmax) was determined by visual estimation from the data plot. Area under the plasma concentration vs. time curve from 0 to the time of the last measurable concentration (AUC) was calculated by the linear trapezoidal method. Elimination half-life (t_{1/2}) was calculated as ln(2)/ke, where ke is the elimination rate constant determined by linear regression of the last three analytically measured points on the plasma concentration vs. time curve.

Body weights of the animal were taken upon arrival, and then monthly. All animals (both left and right eyes) were examined by indirect ophthalmoscopy of the cornea, conjunctiva, anterior chamber, vitreous, posterior chamber, and sclera. One to two drops each of phenylephrine hydrochloride and cyclopentolate hydrochloride were used as mydriatics. Observations on the anterior and posterior segments of the eye were made, graded, and recorded. A modified McDonald-Shadduck scale was used for grading eye irritation and ocular toxicity.

The histopathological processing and evaluation were conducted at Colorado Histo-Prep (Fort Collins, Colo.). Briefly, a central cut of the eye globe was taken, as well as two cuts on either side of the central cut (calottes) at trim. For each eye, the central cut was placed into one cassette, and the two calottes were placed together into a separate cassette. The tissues were processed, embedded in paraffin wax, sectioned by microtome, and stained. Histopathology of the tissues was conducted on slides stained with hematoxylin and eosin. A pathologist who evaluated the tissues had no knowledge of the specific pharmacologic activity or formulation of the test articles. Standardized toxicological pathology criteria and nomenclature for the rabbit were used to categorize microscopic tissue changes.

After single applications of DSP via the non-invasive ocular drug delivery device for 5, 10, or 20 minutes and for all DSP concentrations, significant amounts of DSP and some DEX were found in all the tissues. A typical rank order of DSP amounts in the eye tissue is sclera, conjunctiva, cornea, retina-choroid, anterior chamber, vitreous, and lens. The total amount of drugs in each tissue except vitreous and lens appears to be correlated well with the DSP concentration and application time of the non-invasive ocular drug delivery device. In FIG. 9, the total amount of DSP delivered by the non-invasive ocular drug delivery device was calculated by the sum of DSP and DEX in μg for a purpose of drug delivery analysis. Generally, at a given application duration (i.e., 5, 10, or 20 minutes), a higher DSP formulation concentration yielded a higher amount of DSP in the eye. Similarly, at a given concentration, a longer application duration of the non-invasive ocular drug delivery device yielded a higher amount of DSP in the eye.

The concentration of DSP in each tissue was also calculated in μg/g and summarized in Table 5 for potential efficacy evaluation of the non-invasive ocular drug delivery device. As discussed earlier, the concentration of 1 μg/g or higher in the tissue is considered as a potential therapeutic level. With exception of the lens and vitreous samples in a few cases, most of the ocular tissue concentrations of DSP are significantly higher than 1 μg/g. The typical order of concentration of DSP in ocular tissues, from high to low, was cornea>sclera>conjunctiva>retina-choroid>anterior chamber>lens>vitreous. The drug concentration in the ocular tissues (except lens and vitreous,) correlated well with both increasing DSP concentration in the non-invasive ocular drug delivery device and treatment duration.

TABLE 5
DSP-equivalent concentrations in ocular tissues (mean ± SD, μg/g).

| | | Aqueous | | | Retina- | | |
|---|---|---|---|---|---|---|---|
| Dose | Cornea | Chamber | Lens | Vitreous | Choroid | Sclera | Conjunctiva |
| 4% DSP, | 108 ± 74 | 14 ± 4 | 0 ± 0 | 0 ± 1 | 18 ± 16 | 59 ± 25 | 33 ± 29 |
| 5 min | | | | | | | |
| 4% DSP, | 216 ± 86 | 11 ± 8 | 2 ± 0 | 2 ± 1 | 59 ± 86 | 131 ± 52 | 56 ± 14 |
| 10 min | | | | | | | |
| 4% DSP, | 147 ± 75 | 12 ± 4 | 3 ± 1 | 1 ± 1 | 24 ± 7 | 154 ± 49 | 49 ± 26 |
| 20 min | | | | | | | |
| 8% DSP, | 288 ± 73 | 23 ± 3 | 13 ± 0 | 5 ± 1 | 74 ± 23 | 233 ± 53 | 84 ± 36 |
| 5 min | | | | | | | |
| 8% DSP, | 459 ± 148 | 23 ± 9 | 0 ± 0 | 2 ± 1 | 63 ± 61 | 314 ± 74 | 104 ± 33 |
| 10 min | | | | | | | |
| 8% DSP, | 567 ± 397 | 56 ± 54 | 14 ± 24 | 6 ± 8 | 54 ± 38 | 306 ± 207 | 113 ± 58 |
| 20 min | | | | | | | |
| 15% DSP, | 367 ± 118 | 18 ± 3 | 5 ± 0 | 5 ± 3 | 182 ± 176 | 328 ± 60 | 150 ± 26 |
| 5 min | | | | | | | |
| 15% DSP, | 467 ± 173 | 43 ± 11 | 17 ± 1 | 7 ± 1 | 113 ± 32 | 512 ± 54 | 222 ± 45 |
| 10 min | | | | | | | |
| 15% DSP, | 1128 ± 521 | 89 ± 42 | 13 ± 3 | 12 ± 3 | 351 ± 275 | 615 ± 336 | 287 ± 94 |
| 20 min | | | | | | | |
| 25% DSP, | 714 ± 252 | 39 ± 17 | 6 ± 1 | 6 ± 3 | 114 ± 82 | 452 ± 214 | 221 ± 126 |
| 5 min | | | | | | | |
| 25% DSP, | 512 ± 327 | 35 ± 32 | 1 ± 1 | 4 ± 4 | 60 ± 77 | 429 ± 231 | 184 ± 109 |
| 10 min | | | | | | | |
| 25% DSP, | 2225 ± 886 | 169 ± 67 | 13 ± 4 | 9 ± 4 | 207 ± 101 | 731 ± 189 | 347 ± 107 |
| 20 min | | | | | | | |

Over the course of the 12 week toxicity study entailing 12 weekly doses of DSP via the non-invasive ocular drug delivery device, ocular findings noted with the treated eyes (right eye) were conjunctival injection, discharge, and corneal haze. These ocular findings were transient and mild in nature. No abnormalities or signs of ocular toxicity were observed in untreated eyes (left eye). Details of the ocular findings are given below and a summary of the clinical observations over 12 weeks including the conjunctival injection scores, histopathological results, and body weight is presented in Table 6.

Conjunctiva: Conjunctival injection was generally observed immediately after DSP application in all groups. Resolution period of conjunctival injection correlates with DSP concentration. As the DSP concentration increased, it took longer times to resolve to the baseline. The resolution period of conjunctival injection was generally within 1-2 days for 4% and 8% DSP and up to 7 days for 15% and 25% DSP in some cases. The average conjunctival scores for every 4 weeks indicate that the degree of conjunctival injection increased with the DSP concentration and repeated applications (see Table 6). The animals treated with 4% and 8% DSP had typical conjunctival injection scores immediately after treatment of 1 or <1 through the whole study. In a rare occasion, a score of 2 was found in the 8% DSP group. The animals treated with 15% DSP had typical conjunctival injection scores immediately after treatment of <1 for the first four weeks, and then 2 at Week 8 until the end of study. The animals treated with 25% DSP had typical conjunctival injection scores immediately after treatment of <1 for the first four weeks, and then 2 or 3 at Week 8 until the end of study. Chemosis on the conjunctiva was also observed immediately after DSP administration via the non-invasive ocular drug delivery device. Although chemosis tends to increase in severity with the DSP concentration and with repeated application, the occurrence of chemosis appeared to be sporadic. Conjunctival discharge was noted occasionally but appears to be irrespective of DSP concentration and not related to infection.

TABLE 6
Clinical Observations

| | Average Conjunctival Injection | | | | |
|---|---|---|---|---|---|
| | Score (range) | | | | Ocular |
| | Weeks | Weeks | Weeks | Body | Histopa- |
| Dose | 1-4 | 5-8 | 9-12 | Weight | thology |
| 4% DSP, | 0.11 | 0.25 | 0.31 | NCS | NSF |
| 20 min | (0 to <1) | (0 to 1) | (0 to 1) | | |
| 8% DSP, | 0.11 | 0.19 | 0.40 | NCS | NSF |
| 20 min | (0 to <1) | (0 to 1) | (0 to 2) | | |
| 15% DSP, | 0.27 | 0.72 | 0.93 | 8% loss | NSF |
| 20 min | (0 to 1) | (0 to 2) | (0 to 2) | | |
| 25% DSP, | 0.36 | 1.08 | 1.39 | 13% loss | NSF |
| 20 min | (0 to 1) | (0 to 3) | (0 to 3) | | |

NCS = No clinically significant change.
NSF = No significant findings.

Cornea: Cornea appeared normal after each DSP administration via the non-invasive ocular drug delivery device in all rabbits except in one case with a rabbit in the 15% DSP group from Week 4 to Week 8. Corneal haze on the treated eye was immediately observed in this rabbit after the DSP administration on Week 4. The lesion covered about 40% of the corneal surface. The haze was identified as a result of an off center applicator placement. This caused the drug reservoir to be in direct contact with the cornea during the DSP administration via the non-invasive ocular drug delivery device. The corneal haze grew fainter over time and it was not visible by Week 8.

Body Weight: There were no significant weight changes in the 4% or 8% DSP treated rabbits. However, the animals in the 15% and 25% DSP groups showed trends of decreasing body weight. The consistent decline in body weights of the animals in these two groups indicate that long term exposure at these levels of DSP dosing (i.e., 15% and 25% DSP for 20 min) may have significant systemic side effects on rabbit.

Histopathology: All eyes were considered to be morphologically normal, except one treated eye in the 8% DSP group showed mild chronic inflammation at the limbus of the cornea. Besides that one eye, there were no significant findings (NSF) with any ocular tissue examined. No test article changes were identified.

After single applications of the non-invasive drug delivery device, DSP and DEX were found in plasma for all four treatment regimens (i.e., 5 or 20 minute applications of 4% or 15% of DSP). The plasma concentrations of DSP and DEX after single applications of the non-invasive ocular drug delivery device are shown in FIG. 10a. Tmax of DSP was reached at the first blood draw (5 minutes after device application) whereas Tmax of DEX was reached later at 30 minutes. The maximum plasma concentration (Cmax) of both DSP and DEX increased with increasing DSP concentration and with longer application time. It appears that the concentration affected the systemic exposure more than the application time; the 4% DSP applied for 20 minutes yielded a lower plasma concentration than the 15% DSP applied for 5 minutes. Within 24 hours, the drug plasma concentrations of all groups were approaching or under the lowest detection limit of 1 ng/mL.

For the purpose of assessing the systemic exposure of DSP and DEX, the DSP and DEX plasma concentrations were combined and calculated as DSP equivalent. The DSP equivalent is defined as the sum of DSP and DEX in gram equivalent, with 392.5 g of DEX equivalent to 516.4 g of DSP. The pharmacokinetic profiles of DSP equivalent from all four treatment regimens are shown in FIG. 10b and the key toxicokinetic parameters are presented in Table 7. The half-life of the drug in the rabbit is approximately 2-3 hours, Cmax and AUC increased with increased concentration of DSP and increased application time.

To put the systemic DSP exposure in rabbit into human perspective, estimations of Cmax of the DSP in human were made and presented in Table 7. Cmax values in human were estimated based on Cmax data from IV injections in both rabbit and human: IV injection of 1 mg DSP yields a Cmax of 786 ng/mL in rabbit and 10.5 ng/mL in human. These results suggest that the Cmax of DSP for rabbit is approximately 75 times higher than that for human. The estimated Cmax in human of the lowest dose (4% DSP, 5 minutes) and the highest dose (15% DSP, 20 minutes) of DSP administered via the non-invasive ocular drug delivery device are 2 and 25 ng/mL, respectively.

TABLE 7
DSP-Equivalent Concentrations in Plasma

| | Cₘₐₓ | | AUC | Estimated Cₘₐₓ |
|---|---|---|---|---|
| Dose | (ng/ml) | t_{1/2} (h) | (ng*h/ml) | in Human (ng/ml) |
| 4% DSP, | 148 ± 71 | 3.1 ± 2.2 | 418 ± 93 | 2 ± 1 |
| 5 min | | | | |
| 4% DSP, | 795 ± 344 | 2.3 ± 0.6 | 996 ± 144 | 11 ± 5 |
| 20 min | | | | |
| 15% DSP, | 1188 ± 306 | 1.7 ± 0.9 | 1595 ± 418 | 16 ± 4 |
| 5 min | | | | |
| 15% DSP, | 1844 ± 664 | 2.7 ± 0.3 | 3779 ± 472 | 25 ± 9 |
| 20 min | | | | |

### Example 3—Stability of Dexamethasone Phosphate Composition

Various dexamethasone sodium phosphate (DSP) compositions were prepared at various concentrations and subjected to stability testing conditions. Specific concentrations of DSP tested were 2 wt %, 4 wt %, 8 wt %, and 15 wt %. Otherwise, the composition included water, disodium edetate, and optionally a pH adjuster to achieve a pH of from 6.5 to 7.5. The following tables illustrate some of the stability parameters tested.

TABLE 8
Stability of 2 wt % DSP at 25° C./60% Relative Humidity (RH)

| Specification | T = 0 | 3 Months | 6 Months | 9 Months |
|---|---|---|---|---|
| pH | 7.0 | 7.0 | 7.1 | 7.0 |
| Tonicity | 318 | 319 | 319 | 321 |
| (mOsm/kg) | | | | |
| Assay | 100.7% | 101.1% | 100.1% | 100.2% |
| Dexamethasone | 0.07% | 0.30% | 0.37% | 0.58% |
| Impurity* | | | | |

*Relative to DSP

TABLE 9
Stability of 2 wt % DSP at 2° C. to 8° C.

| Specifications | T = 0 | 6 Months | 12 Months | 18 Months | 24 Months | 36 Months |
|---|---|---|---|---|---|---|
| pH | 7.0 | 7.1 | 7.0 | 7.1 | 7.0 | 7.1 |
| Tonicity | 318 | 318 | 319 | 319 | 316 | 322 |
| (mOsm/kg) | | | | | | |
| Assay | 100.7% | 99.1% | 100.6% | 98.7% | 101.4% | 100.1% |
| Dexamethasone | 0.07% | 0.11% | 0.13% | 0.17% | 0.18% | 0.27% |
| Impurity* | | | | | | |

*Relative to DSP

TABLE 10
Stability of 4 wt % DSP at 25° C./60% Relative Humidity (RH)

| Specification | T = 0 | 3 Months | 6 Months | 9 Months |
|---|---|---|---|---|
| pH | 6.9 | 6.9 | 7.0 | 7.0 |
| Tonicity | 342 | 343 | 343 | 346 |
| (mOsm/kg) | | | | |
| Assay | 102.9% | 101.5% | 100.8% | 101.2% |
| Dexamethasone | 0.07% | 0.34% | 0.42% | 0.64% |
| Impurity* | | | | |

*Relative to DSP

TABLE 11
Stability of 4 wt % DSP at 2° C. to 8° C.

| Specifications | T = 0 | 6 Months | 12 Months | 18 Months | 24 Months | 36 Months | 49 Months |
|---|---|---|---|---|---|---|---|
| pH | 6.9 | 7.0 | 6.9 | 7.0 | 6.9 | 7.0 | 6.6 |
| Tonicity | 342 | 342 | 343 | 344 | 341 | 346 | 345 |
| (mOsm/kg) | | | | | | | |
| Assay | 102.9% | 101.1% | 100.2% | 102.0% | 103.2% | 101.5% | 103.0% |
| Dexamethasone | 0.07% | 0.13% | 0.14% | 0.18% | 0.19% | 0.27% | 0.30% |
| Impurity* | | | | | | | |

*Relative to DSP

TABLE 12
Stability of 8 wt % DSP at 25° C./60% Relative Humidity (RH)

| Specification | T = 0 | 3 Months | 6 Months | 9 Months |
|---|---|---|---|---|
| pH | 7.0 | 6.9 | 6.9 | 7.0 |
| Tonicity | 354 | 356 | 358 | 360 |
| (mOsm/kg) | | | | |
| Assay | 101.7% | 100.9% | 99.1% | 99.5% |
| Dexamethasone | 0.07% | 0.36% | 0.44% | 0.66% |
| Impurity* | | | | |

*Relative to DSP

TABLE 13
Stability of 8 wt % DSP at 2° C. to 8° C.

| Specifications | T = 0 | 6 Months | 12 Months | 18 Months | 24 Months | 36 Months | 49 Months |
|---|---|---|---|---|---|---|---|
| pH | 7.0 | 7.0 | 7.0 | 7.0 | 6.9 | 6.9 | 6.9 |
| Tonicity | 354 | 356 | 355 | 356 | 353 | 359 | 357 |
| (mOsm/kg) | | | | | | | |
| Assay | 101.7% | 100.3% | 101.0% | 100.9% | 101.6% | 100.5% | 101.7% |
| Dexamethasone | 0.07% | 0.12% | 0.14% | 0.19% | 0.20% | 0.32% | 0.33% |
| Impurity* | | | | | | | |

*Relative to DSP

TABLE 14
Stability of 15 wt % DSP at 25° C./60% Relative Humidity (RH)

| Specification | T = 0 | 3 Months | 6 Months | 9 Months |
|---|---|---|---|---|
| pH | 7.0 | 6.9 | 6.9 | 7.1 |
| Tonicity | 576 | 583 | 584 | 590 |
| (mOsm/kg) | | | | |
| Assay | 102.1% | 100.2% | 99.7% | 99.8% |
| Dexamethasone | 0.06% | 0.30% | 0.36% | 0.57% |
| Impurity* | | | | |

*Relative to DSP

TABLE 15
Stability of 15 wt % DSP at 2° C. to 8° C.

| Specifications | T = 0 | 6 Months | 12 Months | 18 Months | 24 Months | 36 Months | 49 Months |
|---|---|---|---|---|---|---|---|
| pH | 7.0 | 7.0 | 6.9 | 7.0 | 6.9 | 6.9 | 6.8 |
| Tonicity | 576 | 582 | 584 | 583 | 576 | 585 | 583 |
| (mOsm/kg) | | | | | | | |
| Assay | 102.1% | 100.2% | 101.3% | 100.7% | 101.9% | 101.0% | 101.4% |
| Dexamethasone | 0.06% | 0.10% | 0.12% | 0.16% | 0.19% | 0.26% | 0.29% |
| Impurity* | | | | | | | |

*Relative to DSP

It should be understood that the above-described methods are only illustrative of some embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, may be made without departing from the principles and concepts set forth herein.

What is claimed is:

1. An ophthalmic composition, comprising:
dexamethasone phosphate, or a salt thereof, present in the composition in an amount from about 1 wt % to about 25 wt %;
dexamethasone present, but in an amount not greater than 1.0 wt % relative to the amount of dexamethasone phosphate, or the salt thereof; and
water,
wherein the composition has a pH of from about 5 to about 8, and
wherein the composition has a tonicity of from about 200 mOsm/kg to about 760 mOsm/kg.

2. The ophthalmic composition of claim 1, wherein the dexamethasone phosphate is dexamethasone sodium phosphate.

3. The ophthalmic composition of claim 1, wherein the dexamethasone phosphate, or the salt thereof, is present in an amount from about 4 wt % to about 18 wt %.

4. The ophthalmic composition of claim 1, wherein dexamethasone is present, but in an amount not more than 1.0 wt % relative to dexamethasone phosphate, or the salt thereof, after storage at ambient temperature for a period of 3 months or less.

5. The ophthalmic composition of claim 1, wherein dexamethasone is present, but in an amount not more than 1.0 wt % relative to dexamethasone phosphate, or the salt thereof, after storage at ambient temperature for a period of 6 months or less.

6. The ophthalmic composition of claim 1, wherein dexamethasone is present, but in an amount not more than 1.0 wt % relative to dexamethasone phosphate, or the salt thereof, after storage at a temperature of from about 2° C. to about 8° C. for a period of 36 months or less.

7. The ophthalmic composition of claim 1, wherein dexamethasone is present, but in an amount not more than 1.0 wt % relative to dexamethasone phosphate, or the salt thereof, after storage at a temperature of from about 2° C. to about 8° C. for a period of 49 months or less.

8. The ophthalmic composition of claim 1, wherein the pH is from about 6.5 to about 7.5.

9. The ophthalmic composition of claim 1, wherein the tonicity is from about 300 mOsm/kg to about 400 mOsm/kg.

10. The ophthalmic composition of claim 1, wherein the tonicity is from about 500 mOsm/kg to about 600 mOsm/kg.

11. The ophthalmic composition of claim 1, wherein the composition is substantially particulate matter free.

12. The ophthalmic composition of claim 1, wherein the composition further comprises a pH adjuster.

13. The ophthalmic composition of claim 1, wherein the composition comprises a chelating agent.

14. The ophthalmic composition of claim 13, wherein the chelating agent is a member selected from the group consisting of: edetate disodium dihydrate, edetic acid, ethylene diamine, porphine, and combinations thereof.

15. The ophthalmic composition of claim 1, wherein the composition does not include a preservative or an antioxidant.

16. The ophthalmic composition of claim 1, wherein the composition does not include a polymer.

17. The ophthalmic composition of claim 1, wherein the composition does not include a cyclodextrin.

18. The ophthalmic composition of claim 1, wherein the composition does not include a surface-active agent.

19. The ophthalmic composition of claim 1, wherein the composition does not include a hydrocarbon.

20. The ophthalmic composition of claim 1, wherein the composition does not include a buffering agent.

21. An ophthalmic system, comprising:
an ophthalmic composition according to claim 1 disposed in a container; and
an ocular drug delivery device configured to couple to an eye of a subject.

22. The ophthalmic system of claim 21, wherein the ophthalmic composition comprises an amount of dexamethasone phosphate, or the salt thereof, of from about 0.1 mg to about 2500 mg.

23. The ophthalmic system of claim 21, wherein the container is a sterile container.

24. The ophthalmic system of claim 23, wherein the sterile container has a volume of from about 0.5 ml to about 10 ml.

25. The ophthalmic system of claim 21, wherein a portion of the ophthalmic composition is preloaded into the ocular drug delivery device.

26. The ophthalmic system of claim 21, wherein the ocular drug delivery device is configured to couple to the eye via negative pressure.

27. A method of treating an ophthalmic condition responsive to dexamethasone phosphate, or a salt thereof, in a subject, comprising:
administering a composition according to claim 1 to an eye of the subject.

28. The method of claim 27, wherein the ophthalmic condition comprises uveitis, age-related macular degeneration (AMD), diabetic retinopathy, diabetic macular edema, dry eye, post-operative inflammation, eye infection, allergic conjunctivitis, corneal trauma, infiltrative keratitis, staphylococcal marginal keratitis, posterior blepharitis, ocular herpetic disease, cystoid macular edema (CME), diabetic retinopathy, Behçet's disease, ocular pain, or a combination thereof.

29. The method of claim 27, wherein administering is performed for a continuous administration period.

30. The method of claim 29, wherein the continuous administration period is from about 1 minute to about 30 minutes.

31. The method of claim 27, wherein administering is performed via an ophthalmic device that is adapted to couple to the eye.

32. The method of claim 31, wherein the ophthalmic device is configured to couple to the eye via negative pressure.

33. The method of claim 27, wherein administering is non-invasive administration.

34. The method of claim 27, wherein administering is passive administration.

35. The method of claim 27, wherein administering is active administration.

36. The method of claim 27, wherein administering delivers a threshold dose of dexamethasone phosphate, or a salt thereof, to the eye of the subject in an amount of from about 0.1 mg to about 10 mg.
